(19) United States
(12) Patent Application Publication
(10) Pub. No.: US 2025/0082211 A1
(43) Pub. Date: Mar. 13, 2025

(54) MULTI-WAVELENGTH PHOTOPLETHYSMOGRAM SYSTEM AND METHOD WITH MOTION ARTIFACT DETECTION

(71) Applicant: GEORGIA TECH RESEARCH CORPORATION, Atlanta, GA (US)

(72) Inventors: Michael CHAN, Atlanta, GA (US); Venu G. GANTI, Atlanta, GA (US); Omer INAN, Marietta, GA (US)

(21) Appl. No.: 18/722,416

(22) PCT Filed: Dec. 20, 2022
(86) PCT No.: PCT/US2022/053507

§ 371 Date: Jun. 20, 2024

Related U.S. Application Data
(60) Provisional application No. 63/291,734, filed on Dec. 20, 2021.

(51) Int. Cl. A61B 5/0205; A61B 5/11; A61B 5/1455; A61B 5/332; A61B 5/339

(52) U.S. Cl. CPC A61B 5/0205; A61B 5/332; A61B 5/1102; A61B 5/14552; A61B 5/339; A61B 2560/0247; A61B 2562/0219; A61B 2562/0238; A61B 2562/06; A61B 2562/227

(57) ABSTRACT

A multi-wavelength PPG system and method configured with optimized mechanical configuration for a chest-worn device for electrocardiogram and multi-wavelength photoplethysmogram acquisition. The multi-wavelength PPG system includes an electrocardiogram sensing assembly having two or more electrodes configured to mount the chest-worn device to the subject's chest. The multi-wavelength photoplethysmogram sensing assembly is located in between the electrocardiogram sensing electrodes to be maintained at the optimal sensing location by the mounting of the electrocardiogram sensing electrodes. The multi-wavelength photoplethysmogram includes one or more additional PPG emitters to provide additional information that can be employed to assess the quality of a signal acquisition with respect to movement artifacts, e.g., to reduce motion artifacts and/or provide accurate target data segments of higher signal quality having improved accuracy for SpO2 estimation.

### RELATED APPLICATION

This PCT application claims priority to, and the benefit of, U.S. Provisional Patent Application No. 63/291,734, filed Dec. 20, 201, entitled “A METHOD TO REMOVE OUTLIERS IN MULTI-WAVELENGTH PHOTOPLETHYSMOGRAM (PPG) USING THE GREEN PPG FOR IMPROVING SpO2 MEASUREMENTS,” which is incorporated by reference herein in its entirety.

### STATEMENT OF GOVERNMENT INTEREST

This invention was made with government support under grant number 75D30120C09558, awarded by the Center for Disease Control. The government has certain rights in the invention.

### BACKGROUND

Photoplethysmogram may be acquired by emitting light onto the skin and sensing the reflected light to assess the movement of blood flow in tissue. When two light sources of different wavelengths are employed, in which one wavelength is absorbable by a compound of interest, e.g., deoxygenated hemoglobin, while the other one does not, the two sensed measurements could provide measures parameters such as oxygen saturation (or the concentration of oxygenated hemoglobin). Pulse oximetry operates in such ways.

Portable, long-term, continuous pulse oximetry, e.g., via wearable devices, has utility in clinical settings as well as for everyday Internet of Things (IoT) wearable devices such as smart watches. Pulse oximeters or photoplethysmography may be used to assess health parameters in combination with other sensors. The collecting of high-quality data remains challenging due to motion artifacts, including those for chest-worn devices.

There is a benefit to improving biophysical signal acquisition.

### SUMMARY

A multi-wavelength PPG system and method are disclosed configured with optimized mechanical configuration for a chest-worn device for electrocardiogram and multi-wavelength photoplethysmogram acquisition. The multi-wavelength PPG system includes an electrocardiogram sensing assembly having two or more electrodes configured to mount the chest-worn device to the subject's chest. The multi-wavelength photoplethysmogram sensing assembly is located in between the electrocardiogram sensing electrodes to be maintained at the optimal sensing location by the mounting of the electrocardiogram sensing electrodes. The multi-wavelength photoplethysmogram includes one or more additional PPG emitters to provide additional information that can be employed to assess the quality of a signal acquisition with respect to movement artifacts, a common issue for pulse oximetry. The exemplary multi-wavelength photoplethysmogram can reduce motion artifacts to provide accurate target data segments of higher signal fidelity or quality, having improved accuracy for SpO2 estimation.

The wavelengths for photoplethysmogram may be of any wavelength as described or referenced herein.

In some embodiments, the system employs a third PPG signal (e.g., having a different wavelength from those used for pulse oximetry) to generate a signal that can be used to create a template for rejecting PPG waveforms of the other two wavelengths having lower-quality signals due to potential motion artifact contamination. Because the PPG signals in different wavelengths have different penetration depths, the exemplary acquisition would have different levels of robustness against motion artifacts.

In some embodiments, estimation of SpO2 and perfusion index (PI) is performed by extracting an alternating current (AC) and the direct current (DC) component of two of the PPG signals. The pulsatile, small-signal AC component of each wavelength shows different levels of signal quality. The green AC component of PPG has the highest signal quality, followed by the infrared and red AC components of PPG. These AC components may differ in morphologies due to their differences in penetration depths and absorption coefficients without preprocessing their signals. However, after filtering them using a bandpass filter, these signals can exhibit similar morphologies. A template signal can be generated using the green PPG, and the other two PPG signals in different wavelengths can be compared against this template to determine the signal quality index (SQI). The SQI may be computed using normalized cross-correlation between the template and the test PPG signal. The proposed method can ensure that only high-quality PPG signals are used for estimating SpO2.

Exemplary systems and methods are disclosed for a chest-wearable device (e.g., wearable patch) that can be used to obtain Photoplethysmogram waveform acquisitions that can be employed to determine a peripheral oxygen saturation estimation or to diagnose or evaluate cardiac health.

In some embodiments, a chest-wearable device is provided. The chest-wearable device comprises a device body having an underside surface; at least one electrocardiogram (ECG) electrode interface, including a first ECG electrode interface and a second ECG electrode interface, wherein the first ECG electrode interface is located at a first location on the underside surface of the device body, and the second ECG electrode interface is located at a second location on the underside of the device body, each of the first ECG electrode interface and the second ECG electrode interface is configured to operatively couple to a respective patch electrode; at least one optical sensor (e.g., photodiode) configured to obtain Photoplethysmogram waveform acquisitions; and a plurality of emitters, including a first emitter, a second emitter, and a third emitter. In some embodiments, the plurality of emitters is configured to emit light at a person to be received by at least one optical sensor. In some embodiments, each of the first emitter and the second emitter is configured to operate at a different wavelength configuration for the Photoplethysmogram waveform acquisitions, and the third emitter is employed for artifact identification. In some embodiments, the first emitter, the second emitter, and the third emitter are each positioned at a respective location on the underside surface between the first ECG electrode interface and the second ECG electrode interface. In some embodiments, the first ECG electrode interface and second ECG electrode interface, when attached to the respective patch electrodes that are attached to a person, position the first emitter, the second emitter, and the third emitter proximal to the person for the Photoplethysmogram waveform acquisition.

In some embodiments, at least one of the first emitter, the second emitter, the third emitter, and the at least one optical sensor extends from the underside to position proximally to the person for the Photoplethysmogram waveform acquisition.

In some embodiments, the chest-wearable device further comprises: a controller configured, via computer readable instructions or electronic circuitries, to (i) generate a signal quality template of output signals acquired from the third emitter, (ii) generate a signal quality index for a first output signal associated with the first emitter and/or a second output signal associated with the second emitter, wherein the signal quality index for the each of the first and second output signals are employed to reject a portion of the output signals from a peripheral oxygen saturation estimation.

In some embodiments, the chest-wearable device further comprises: a controller configured, via computer readable instructions or electronic circuitries, to (i) generate a signal quality template of output signals acquired from the third emitter, (ii) generate a signal quality index for a first output signal associated with the first emitter and/or a second output signal associated with the second emitter, wherein the signal quality index for the each of the first and second output signals are employed to reject a portion of the output signals from a clinical analysis employing the first output signal and the second output signal (e.g., to determine an assessment of heart health).

In some embodiments, the chest-wearable device further comprises: a controller configured, via computer readable instructions or electronic circuitries, to (i) determine a weight vector of output signals associated with the first, second, and third emitters and (ii) compute a reference signal as a motion-robust AC component for (1) peripheral oxygen saturation estimation or (2) analysis employing the first output signal and the second output signal. In some embodiments, the weight vector is determined via a matrix operation.

In some embodiments, the first emitter has a center wavelength around a red spectrum.

In some embodiments, the second emitter has a center wavelength around an infrared spectrum.

In some embodiments, the third emitter has a center wavelength around a green spectrum.

In some embodiments, the Photoplethysmogram waveform acquisitions are employed to determine a peripheral oxygen saturation estimation.

In some embodiments, the chest-wearable device further comprises an output display or wireless interface to display, at the output display or an external display, the peripheral oxygen saturation estimation.

In some embodiments, the output display or the wireless interface is configured to display, at the output display or the external display, the electrocardiogram waveform acquisitions from the first ECG electrode interface and the second ECG electrode interface.

In some embodiments, the chest-wearable device further comprises a seismographic sensor configured to measure the seismocardiogram signal of the person.

In some embodiments, the chest-wearable device further comprises a controller configured to determine peripheral oxygen saturation estimation as a ratio of identified alternating current (AC) components and identified direct current (DC) components of the first and second emitter.

In some embodiments, the at least one optical sensor comprises a first optical sensor, wherein the first optical sensor is positioned at the underside surface and proximal to the plurality of emitters.

In accordance with certain embodiments, a method to determine pulmonary wedge pressure (PCWP), and pulmonary artery pressure (PAP) using the chest-wearable device is provided.

### BRIEF DESCRIPTION OF THE DRAWINGS

The skilled person in the art will understand that the drawings described below are for illustration purposes only.

FIGS. 1A and 1B each show an example multi-wavelength PPG device in accordance with an illustrative embodiment.

FIGS. 2A and 2B each provide a respective view of an example device in accordance with an illustrative embodiment.

FIG. 3A shows an example signal processing pipeline for pulse oximetry estimation operation in accordance with an illustrative embodiment.

FIG. 3B shows an example operation 310 to perform outlier rejection using signal SQI derived from a third PPG signal in accordance with an illustrative embodiment.

FIG. 4 is a schematic diagram of a breath hold study that employs the multi-wavelength PPG device of FIG. 1A in accordance with an illustrative embodiment.

FIG. 5 is a schematic diagram illustrating Biopac signals and biosensor signals acquired during the breath-hold study (e.g., during a fourth breath-hold and during normal breathing).

FIG. 6 is a schematic diagram illustrating an example signal processing pipeline employed in the study for pulse oximetry estimation operation in accordance with an illustrative embodiment.

FIG. 7 shows different model training schemes for computing the pulse oximetry estimate in accordance with some illustrative embodiments.

FIGS. 8A-8F show regression plots and Bland Altman plots that demonstrate estimation results for different calibration and estimation schemes.

FIG. 9 is a graph showing the results of different calibration methods.

FIGS. 10A and 10B are graphs showing the results of calibration analysis.

FIG. 11 is a graph showing Mean bias across varying Fitzpatrick skin types.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

### Example Device

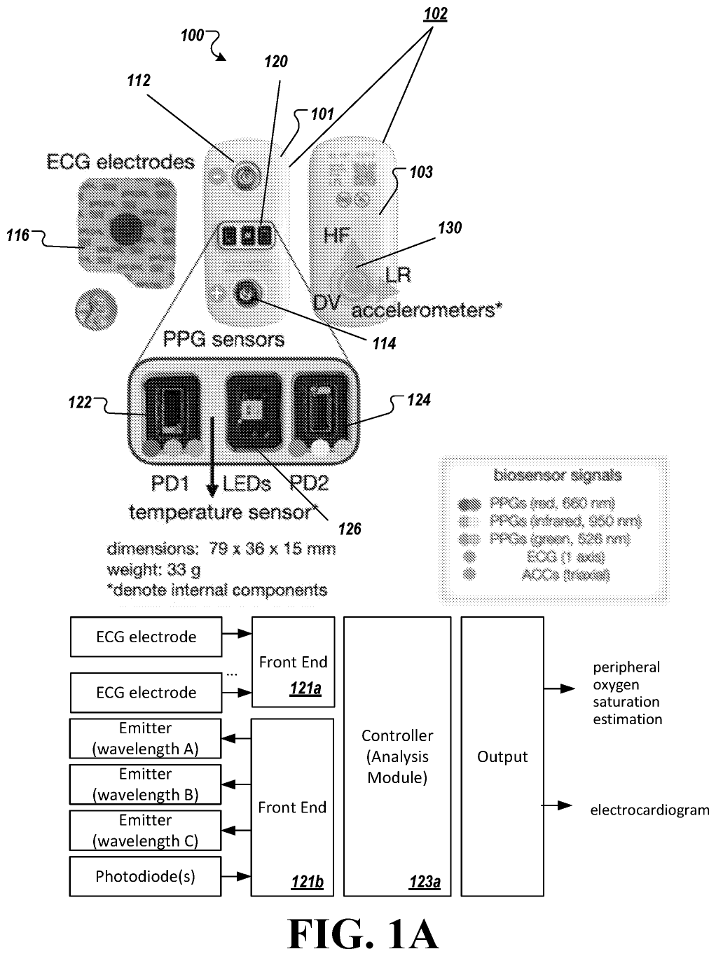

FIG. 1A is a schematic diagram 100 depicting views of an example device 102 (e.g., chest-wearable device, wearable patch biosensor) according to an illustrative embodiment. In the example shown in FIG. 1A, the multi-wavelength PPG system 102 is configured with an optimized mechanical configuration for a chest-worn device for electrocardiogram and multi-wavelength photoplethysmogram acquisition. The multi-wavelength PPG system 102 includes an electrocardiogram sensing assembly having two or more electrodes (116) configured to mount the chest-worn device to the subject's chest. A multi-wavelength photoplethysmogram sensing assembly is located in between the electrocardiogram sensing electrodes to be maintained at the optimal sensing location by the mounting of the electrocardiogram sensing electrodes. The multi-wavelength photoplethysmogram includes one or more additional PPG emitters to provide additional information that can be employed to assess the quality of a signal acquisition with respect to movement artifacts, a common issue for pulse oximetry. The exemplary multi-wavelength photoplethysmogram can reduce motion artifacts to provide accurately target data segments of higher signal quality, having improved accuracy for SpO2 estimation.

Integrated ECG Acquisition and Mounting. In particular, FIG. 1A shows the device 102 with an underside surface 101 opposite a top surface 103. The underside surface 101 includes integrated ECG and PPG electrodes, including (i) a first ECG electrode interface 112 and a second ECG electrode interface 114 that is configured to couple to removable ECG pads 116 to obtain ECG signals and (ii) a PPG assembly to obtain multi-wavelength PPG, from a wearer of the device 102. As shown, the first ECG electrode interface 112 is located at a first location on the underside surface 101, and the second ECG electrode interface 114 is located at a second location on the underside surface 101 of the device 102. Each of the first ECG electrode interface 112 and the second ECG electrode interface 114 are configured to operatively couple to a respective patch electrode 116 that can be affixed to the wearer's skin. The respective patch electrodes 116 are formed or attached to a surface of the device 102 and operate as an electrode of the sensor and a connecting pad for an integrated sensor that can contact or be in proximity to the skin. The patch electrodes 116 of the device 102 utilize the ECG electrode interfaces 112, 114 to ensure close contact between the skin and the sensors (e.g., at least one optical sensor 120), which is necessary for high-fidelity ECG and PPG acquisition.

As depicted in FIG. 1A, the underside surface 101 of the device 102 comprises at least one optical sensor 120 (e.g., photodiode) positioned proximal to a plurality of emitters 126 (e.g., Light Emitting Diodes (LEDs)), including a first emitter, a second emitter, and a third emitter. The first emitter and the second emitter are each configured to operate at a different wavelength configuration for the Photoplethysmogram waveform acquisitions, for example, to determine a peripheral oxygen saturation estimation. In some implementations, the third emitter is employed for artifact identification. For example, the first emitter may have a center wavelength around a red spectrum, the second emitter may have a center wavelength around an infrared spectrum, and the third emitter may have a center wavelength around a green spectrum.

As depicted in FIG. 1A, the example device 102 defines a body having a substantially elliptical shape. In other examples, the example device 102 may comprise other shapes including, but not limited to, a hexagonal, circular, or rectangular shape. In various embodiments, the device 102 is configured to be worn proximal to a person or wearer (e.g., disposed on or affixed to a chest area) to facilitate and electrocardiogram and photoplethysmogram waveform acquisition. In the example shown in FIG. 1, the device 102 is configured to be placed at the sternum to provide accurate SpO2 measurements at the chest.

In some embodiments, the plurality of emitters 126 may form or define an array. The plurality of emitters 126 are configured to emit a light beam at a person (e.g., to a surface of a wearer's skin) that is, in turn, received by the at least one optical sensor 120. In the example shown in FIG. 1A, the optical sensors include a first optical sensor 122 (e.g., photodiode) and a second optical sensor 124 (e.g., photodiode) positioned on either side of the plurality of emitters 126.

Multi-Wavelength PPG. In various implementations, the device 102 includes emitters (e.g., LEDs) of multiple wavelengths (e.g., three or more, e.g., red, infrared, and green) that are configured as a photoplethysmogram sensing assembly that operates with the ECG electrode (e.g., 112, 114). The multi-wavelength photoplethysmogram sensing assembly is located in between the electrocardiogram sensing electrodes to be maintained at the optimal sensing location by the mounting of the electrocardiogram sensing electrodes (e.g., 112, 114). The multi-wavelength photoplethysmogram may employ conventional two-channel PPG waveform acquisitions and further includes one or more additional PPG emitters to provide additional information that can be employed to assess the quality of a signal acquisition with respect to movement artifacts, a common issue for pulse oximetry.

The additional wavelength may be employed in subsequent processing for outlier rejection or motion robustness assessment as further described herein. In FIG. 1A, the device 102 includes the analog-front-end circuits (shown as “Front End” 121a, 121b) for measuring ECG signals and PPG signals.

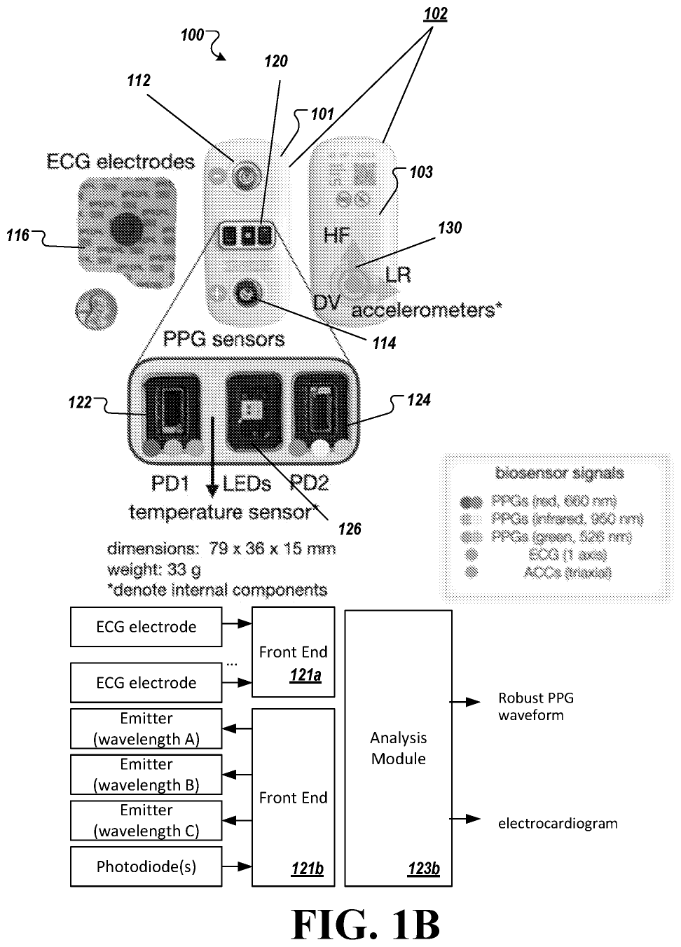

Analysis Module. In FIGS. 1 and 2, the front-end electronics (121a, 121b) are coupled to a controller 123 (shown as 123a and 123b) comprising one or more analysis module that is configured to provide (i) a peripheral oxygen saturation estimation or a PPG waveform, and (ii) an electrocardiogram. In FIG. 1A, the front-end electronics are coupled to analysis module 123a to provide a peripheral oxygen saturation estimation and (ii) an electrocardiogram. In FIG. 1B, the front-end electronics 121a, 121b are coupled to an analysis module 123b that is configured to provide a PPG waveform that can be used for the assessment of cardiac health. Examples of assessment of cardiac health employing PPG waveform and electrocardiogram are provided in PCT Patent Publication No. WO2022/115228, which is incorporated by reference herein.

Additionally, in various embodiments, the device 102 comprises a controller. The controller may be similar or identical to the computing device described herein in relation to Fig. X. In the example described above, where the plurality of emitters 126 comprises three emitters, the controller is configured, via computer readable instructions or electronic circuitries, to generate a signal quality template of output signals acquired from the third emitter and generate a signal quality index for a first output signal associated with the first emitter and/or a second output signal associated with the second emitter. The signal quality index for the each of the first and second output signals can be employed to reject a portion of the output signals from a peripheral oxygen saturation estimation, is discussed in more detail herein. In some embodiments, the controller is configured to determine a peripheral oxygen saturation estimation as a ratio of identified alternating current (AC) components and identified direct current (DC) components of the first and second emitter.

In some embodiments, the controller is further configured, via computer readable instructions or electronic circuitries, to generate a signal quality template of output signals acquired from the third emitter and generate a signal quality index for a first output signal associated with the first emitter and/or a second output signal associated with the second emitter. The signal quality index for each of the first and second output signals can be employed to reject a portion of the output signals from a clinical analysis employing the first output signal and the second output signal (e.g., to determine an assessment of heart health).

In some embodiments, the controller is further configured, via computer readable instructions or electronic circuitries, to determine a weight vector of output signals associated with the plurality of emitters 126 (e.g., the first emitter, the second emitter, and the third emitter) and compute a reference signal as a motion-robust AC component for peripheral oxygen saturation estimation or analysis employing the first output signal and the second output signal. In some embodiments, the weight vector is determined via a matrix operation.

Example Health Parameter Assessment System. In accordance with certain embodiments of the present disclosure, methods for determining pulmonary wedge pressure (PCWP) and/or pulmonary artery pressure (PAP), for example, using the device 102 described above in connection with FIG. 1A, are provided herein. As noted above, the device 102 can comprise one or more sensors (e.g., an accelerometer, seismocardiogram sensor, or other sensors) that can be used to measure a seismocardiogram (SCG) signal of a person.

For example, the sensor (e.g., accelerometer) can be configured to measure tri-axial SCG signals. For example, tri-axial SCG signals can include the dorso-ventral, lateral, and/or head-to-foot axis. Alternatively, or in addition, the sensor (e.g., accelerometer) can be configured to measure a gyrocardiogram signal of the user.

An example method can include receiving data from a sensor relating to the cardiogenic vibrations of a person. The method can include determining, based on data from the sensor, the filling characteristics of the heart. For example, the filling characteristics of the heart can be based at least in part one or more axes of a seismocardiogram signal (e.g., lateral, heat-to-foot, dorso-ventral). Alternatively, or in addition, the filling characteristics can be based, at least in part, on the seismocardiogram signal during a diastolic portion of a heartbeat.

Baseline pulmonary pressure values, as well as the changes in pressure, can be tracked using features from noninvasive SCG and ECG signals using population-level regression algorithms. An unobtrusive device and corresponding signal processing and machine learning algorithms capable of tracking the filling pressure of the heart via measuring the pulmonary pressures can enable remote home monitoring for patients with cardiovascular diseases. The device 102 has a unique combination of ECG, SCG, and environmental sensing capabilities, which allows for context-aware determination of hemodynamic parameters in unsupervised settings. Additionally, the measurement of environmental parameters such as altitude, humidity, and temperature allows for the estimated hemodynamic parameters such as pulmonary capillary wedge pressure (PCWP) or pulmonary artery pressure (PAP) to be put in the context of activity and/or environment. For example, vasodilation in the heat will lead to relative hypovolemia in the cardiovascular system, thus resulting in a decrease in preload (decrease in PCWP and PAP); adding these environmental features together with the SCG and ECG features for the machine learning-based model estimating hemodynamic parameters will be more accurate.

Accelerometer Acquisition system. As further depicted in FIG. 1A, at least one accelerometer 130 is located on the top surface 103 of the device 102. The accelerometer 130 may be configured to obtain movement data that can be used to measure a seismocardiogram signal of a person and/or determine a pulmonary wedge pressure of a person. In various implementations, other types of sensors may be integrated into the device, including, for example, without limitation, a capacitor, a temperature sensor, environmental sensor, humidity sensor, vibration sensor, pressure sensor, position sensor, combinations thereof, and/or the like.

In some embodiments, the device 102 further comprises an output display or wireless interface to display, at the output display or an external display, the peripheral oxygen saturation estimation determined via the controller of the device 102. In some embodiments, the output display or the wireless interface is configured to display, at the output display or the external display, the electrocardiogram waveform acquisitions from the first ECG electrode interface 112 and the second ECG electrode interface 114.

Example Placement. FIG. 2A and FIG. 2B are views 201 and 203, respectively, of the example device 102 as placed on the sternum according to illustrative embodiments. In FIG. 2A, From a frontal view 201, the device 102 (e.g., chest-wearable device, wearable patch biosensor) is attached to the sternum of the subject using ECG electrode interfaces (such as but not limited to the ECG electrode interfaces 112, 114 described above in connection with FIG. 1A) and/or patch electrodes (such as but not limited to the patch electrodes 116 described above in connection with FIG. 1A) that are located on an underside surface of the device 102. As depicted in FIG. 2A, the superior end (e.g., top edge) of the device 102 starts approximately two fingers down from the suprasternal notch 204.

Referring now to FIG. 2B, from a lateral view 203, the protruding part of the device 102 that houses the plurality of emitters (e.g., LEDs) and one or more optical sensors (e.g., photodiodes) can be seen. As depicted in FIG. 2A and FIG. 2B, the housing of the PPG sensors (including emitters (e.g., LEDs) and optical sensor(s) (e.g., photodiodes) protrudes out of the patch electrode 205. Because of this mechanical design, the PPG sensors apply pressure on the sternal skin and help the device 102 (e.g., via the patch electrode 205) remain in close contact with the skin.

### Example Peripheral Oxygen Saturation (SpO2) Estimation

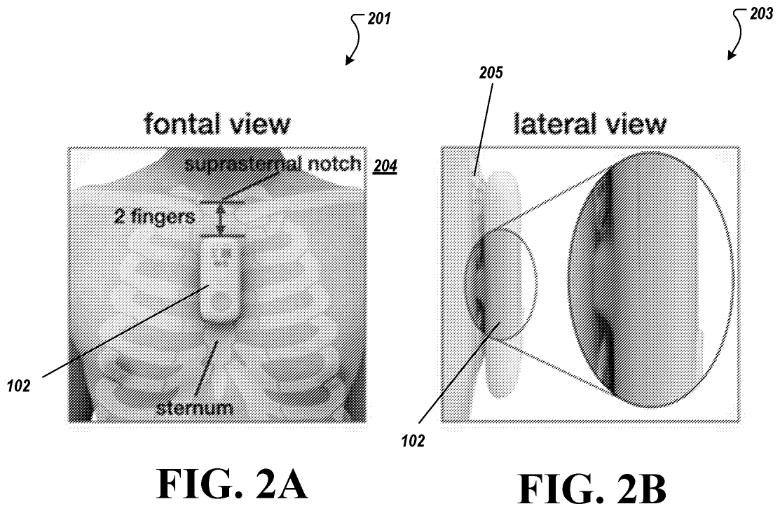

FIG. 3A shows an example signal processing pipeline 300, e.g., for operation by the analysis module 123, for pulse oximetry estimation operation. The ECG may be used to localize specific segments of the PPG signals (e.g., R-peaks) to better extract SpO2-related features. In the example shown in FIG. 3A, the analysis module 123 is configured to determine the peripheral oxygen saturation (SaO₂) as a ratio of the normalized AC component (also a ratio) of two optical wavelengths from the PPG signals per Equation 1.

$$R = \frac{A {C}_{red}}{A {C}_{I R}} / \frac{D {C}_{red}}{D {C}_{I R}} \qquad ( Equation l )$$

In Equation 1, AC_{red} is the AC component of the one of the PPG signals, e.g., red PPG, AC_{IR} is the AC component of the second PPG signal, e.g., IR PPG, DC_{red} is the DC component of the first PPG signal, i.e., red PPG, and DC_{IR} is the DC component of the second PPG signal, i.e., IR PPG. The analysis module 123 may use R, along with the absorption coefficients of oxyhemoglobin (HbO₂) and deoxyhemoglobin (Hb) for different wavelengths, to derive SaO₂ directly. The relationship between SaO₂ and R may be defined per Equation 2:

$${SaO}_{2} = \frac{{ε}_{H b} ( {λ}_{red} ) - {ε}_{H b} ( {λ}_{I R} ) R}{{ε}_{H b} ( {λ}_{red} ) - {ε}_{H b {O}_{2}} ( {λ}_{red} ) + [ {ε}_{H b {O}_{2}} ( {λ}_{I R} ) - {ε}_{H b} ( {λ}_{I R} ) ] R} \qquad ( Equation 2 )$$

In Equation 2, ε_{Hb} is the absorption coefficient of Hb, ε_{HbO2} is the absorption coefficient of HbO₂, λ_{red} is the wavelength of the red PPG, and λ_{IR} is the wavelength of the IR PPG. Equation 2 can be further approximated using a Taylor series expansion as Equation 3.

$$= \frac{A \times R + B}{C \times R + D} \approx m \times R + b = {SpO}_{2} \qquad ( Equation 3 )$$

Equation 3 defines an empirical model that governs the relationship between SpO2, the surrogate of SaO₂, and R. In Equation (3), parameters A, B, C, and D can replace the absorbance coefficients of the Hb and HbO₂ at the two wavelengths, m is the slope, and b is the intercept. To determine the ratio of the AC to DC components, the analysis module 123 may perform a beat segmentation operation (306) and an R-peak detection operation (308). Additional descriptions of the PPG estimation may be found in [17], which is incorporated by reference herein.

### Third PPG Signal-Based Outlier Rejection Module Example #1

As shown in FIG. 3A, the peripheral oxygen saturation (SaO₂) may be determined as a ratio of the normalized AC component (also a ratio) of two optical wavelengths from the PPG signals per Equation 1. This type of operation, among other operations, may be susceptible to noise or motion artifacts.

To improve signal fidelity and reduce effects from motion artifacts and involuntary respiratory movements that can reduce the accuracy of SpO₂ estimation, the analysis module 123 may additionally perform an outlier rejection operation, e.g., via Module 310 using the third PPG signal (e.g., green PPG signal), to calculate a “signal-quality-index” (SQI) for the two PPG signals used to determine the peripheral oxygen saturation. The PPG signal SQI may be determined by computing a signal quality template and comparing it against the other PPG signals or a parameter derived therefrom.

FIG. 3B shows an example operation 310 to perform outlier rejection using signal SQI derived from a third PPG signal. In the example shown in FIG. 3B, the outlier rejection module 310 includes a template creation module 312 and a signal SQI module 314. The signal (SQI) module 314 may perform an empirically determined threshold (e.g., 0.7) and is configured to exclude a sample is excluded if either SQI method suggests so.

To determine the similarity, the analysis module 123 is configured, per the temperate creation module 312, in some embodiments, to normalize cross-correlation (NCC) between a PPG beat with its corresponding template, e.g., per Equation 4.

$$N C {C}_{k , λ} = \frac{{Σ}_{n = 1}^{N} ( {PPG}_{λ} ( n ) - \overline{PP {G}_{λ}} ) ( PP {G}_{g r e e n} ( n + k ) - \overline{PP {G}_{g r e e n}} )}{\sqrt{{{Σ}_{n = 1}^{N} ( {PPG}_{λ} ( n ) - \overline{PP {G}_{λ}} )}^{2} {{Σ}_{n = 1}^{N} ( P P {G}_{g r e e n} ( n + k ) - \overline{PP {G}_{g r e e n}} )}^{2}}} \qquad ( Equation 4 )$$

The outlier rejection module 310 may then define and reject outliers in the AC ratios that deviate by a pre-defined deviation (e.g., three, four, five, six, etc., median absolute deviations from the median of two or three of the signals), per the signal SQI module 314.

In Equation 4, PPG_{λ}(n) denotes the n^{th} sample in the PPG beat of wavelength λ, P̅P̅G̅λ̅ denotes the average value of the samples in a PPG beat of wavelength λ, and NCC_{k, 2} denotes the correlation coefficient between PPG_{λ} and the k-lag PPG_{green}. The parameters may be determined from the beat segmentation and AC/DC extraction operation 306, shown comprising a bandpass filter 306a, a lowpass filter 306b, and a segmentation and beat ensemble averaging 306c. Next, the maximal NCC_{λ}, NCC_{max,λ}, may be defined as per Equation 5.

$$N C {C}_{max , λ} = {arg max}_{k} {NCC}_{k , λ} \qquad ( Equation 5 )$$

In Equation 5, NCC_{max,λ} is employed as a measure of the SQI of the PPG_{λ} and has a range of [0, 1]. Other ranges may be employed. As shown in FIG. 3B, the SQI for the DC_{IR} and DC_{RED} may be evaluated to determine if time or data regions associated with low SQI values (e.g., threshold of 0.7) are present; the low SQI regions may be excluded from peripheral oxygen saturation estimation or

### Third PPG Signal-Based Outlier Rejection Module Example #2

To improve signal fidelity and reduce effects from motion artifacts and involuntary respiratory movements, the analysis module 123 may additionally (or in combination with the SQI module) perform a relative pulsatility analysis of the color channels in the three or more PPG signals. By using an additional third wavelength in the device as described herein, the analysis module 123 can determine a motion-free signal \vec{P_{bv}} and pulse-signal \vec{S}. The pulse-signal \vec{S} may be correlated with the motion-corrupted Cₙ matrix to equal the motion-free signal \vec{P_{bv}} and may be directly used as a reference signal to compute a motion-robust AC component, e.g., as employed in operation described in relation to FIG. 3A.

The pulse-signal S may be determined per Equation 6 where the weight vector {right arrow over (\vec{W_{PBV}})}∈ [symbol] ^{1×3}.

$$\vec{S} = \vec{{W}_{P B V}} {C}_{n} \qquad ( Equation 6 )$$

In Equation 6, Cₙ is a motion-corrupted matrix, and \vec{W_{PBV}} is a weight vector that can be computed as {right arrow over (\vec{W_{PBV}})}=k\vec{P_{bv}} Q⁻¹, where Q⁻¹=CₙCₙ^{T}. The motion-free signal \vec{P_{bv}} can be determined as the correlation of \vec{S} and Cₙ^{T}, S\vec{C}ₙ^{T}=k\vec{P_{bv}}, where k is the normalizing vector, so the norm of \vec{P_{bv}} is 1.

Relative pulsatility analysis can operate on the basis that the relative pulsatility of the color channels in PPG remain relatively constant across subjects of different skin melanin level [41]. The relative pulsatility analysis may evaluate the signature vector of the blood volume change that can capture the relative ratio of the pulsatility across the multi-wavelength channels. Mathematically, the signature vector P_{bv} can be defined as P_{bv}∈ [symbol] ^{1×3}. In one embodiment, the relative pulsatility analysis determines the signature vector based on a measure of each combination of the multi-wavelength channels of the sensing system. The combination of the multi-wavelength channels of the sensing system may be used in a calibration procedure, which may be used to provide parameters for the relative pulsatility analysis. For example, while the signature vector P_{bv} of the PPG channels may have different values in the vector elements, e.g., P_{bv}=[0.39, 0.70, 0.60], motion-induced distortion (denoted Pₘₒₜᵢₒₙ) would exhibit itself equally in all color channels, e.g., Pₘₒₜᵢₒₙ=[0.58, 0.58, 0.58].

The relative pulsatility analysis is configured to employ the three PPG signals (e.g., red, infrared, and green PPG signal) with a fixed duration, e.g., \vec{R}, \vec{I}, and \vec{G} (\vec{R}, \vec{I}, and \vec{G}∈ [symbol] ^{1×T}). The relative pulsatility analysis determines the respective mean-centered normalized multi-wavelength signals as \vec{Rₙ}, \vec{Iₙ}, and \vec{Gₙ} and concatenate the three vectors to form Cₙ (Cₙ ∈ [symbol] ^{3×T})

The derivation of the analysis may first compute the correlation of \vec{S} and Cₙ^{T} to obtain \vec{P_{bv}}, \vec{S}Cₙ^{T}=k\vec{P_{bv}}, where k is the normalizing vector, so the norm of \vec{P_{bv}} is 1. If the analysis then replaces \vec{S} by {right arrow over (\vec{W_{PBV}})}Cₙ, the analysis can obtain the following relationship, {right arrow over (\vec{W_{PBV}})} CₙCₙ^{T}=k\vec{P_{bv}}. After rearranging the terms and isolating {right arrow over (\vec{W_{PBV}})}, the following parameter can be determined where \vec{W_{PBV}}=k\vec{P_{bv}}Q⁻¹, where Q⁻¹=CₙCₙ^{T}. Since \vec{P_{bv}} (per above) and Cₙ (per above) are known, \vec{W_{PBV}} can be computed by a straightforward matrix operation. With {right arrow over (\vec{W_{PBV}})}, the analysis can compute \vec{S}, which contains only the signal with variations induced by materials that give rise to the expected \vec{P_{bv}}. As noted above, the \vec{S} can be used as a reference signal to compute a motion-robust AC component.

### EXPERIMENTAL RESULTS AND EXAMPLES

A study was conducted that developed an accurate SpO2 estimation using a custom chest-based wearable patch biosensor capable of measuring electrocardiogram (ECG) and photoplethysmogram (PPG) signals with high fidelity, as described herein. Through a breath-hold protocol, the study collected physiological data with a wide dynamic range of SpO2 from 20 subjects. The ratio of ratios (R) used in pulse oximetry to estimate SpO2 was robustly extracted from the red and infrared PPG signals during the breath-hold segments using feature extraction and PPG_{green}-based outlier rejection algorithms, described in relation to FIG. 3A. Through subject-independent training, the study achieved a low root-mean-square error (RMSE) of 2.64±1.14% and a Pearson correlation coefficient (PCC) of 0.89. With subject-specific calibration, the study further reduced the RMSE to 2.27±0.76% and increased the PCC to 0.91. In addition, the study showed that calibration is more efficiently accomplished by standardizing and focusing on the duration of breath-hold rather than the resulting range in SpO2. The accurate SpO2 estimation provided by the custom biosensor and the algorithms provide research and clinical opportunities for a wide range of disease and wellness monitoring applications.

The study demonstrated that the custom, chest-worn wearable patch biosensor was capable of accurately estimating SpO2 while subjects underwent a 10 breath-hold protocol. It was observed that standardizing the calibration duration, rather than the calibration range, was the most important factor for optimal calibration. Finally, it was observed that differences in Fitzpatrick skin types do not introduce disparities in bias. Together with its holistic cardiac monitoring, this device can provide longitudinal and quantitative information of disease progression in both cardiovascular and pulmonary diseases.

Materials and Methods—Principle of PPG. The study used a PPG signal that represents the changes in the reflected light emitting diodes' (LEDs) light intensity, as detected by the photodiodes (PDs). According to the Beer-Lambert law, the intensity of the reflectance PPG measured is related to the optical path length of light traveled from the LEDs to the PDs (pp. 47-48). The changes in PPG intensity with respect to each component (arterial blood, venous blood, tissue, bone, etc.) have different pulsating dynamics [20]. By using appropriate filter banks, the cardiac pulsation of the PPG is leveraged to target its arterial, pulsatile, small-signal component. Specifically, the portion of the PPG signal that is representative of cardiac pulsation and the periodic changes in blood volume is termed the alternating current (AC) component, and the baseline wander of the PPG-which is slower than the cardiac frequency—is termed the direct current (DC) component [20]. The AC and DC components of the PPG in multiple wavelengths (i.e., red and IR PPG) can reveal the oxygenation saturation of the underlying arteries [17]. More details are provided herein.

Breath-Hold Study Design. The breath-hold study was designed to induce hypoxemia and sufficient changes in SpO2. This study was conducted under a protocol approved by the Georgia Institute of Technology Institutional Review Board (H21100). A total of 22 (16 males, 6 females) young volunteers were recruited for the breath-hold study, and written informed con-sent was obtained. The number of subjects recruited exceeds that of similar studies [14,21,22]. In this dataset, two subjects were excluded from the analysis. The data of one subject suffered from poor ECG quality—due to expired ECG electrodes that were in-advertently used. The data of the subject exhibited an abnormal distribution of the extracted features compared to those shown in (p. 51). Specifically, the ratio of ratios (R) systematically deviates more than three standard deviations across all SpO2 levels. Therefore, for this work, only data of the remaining 20 subjects were used for analysis. Demographic information of these 20 subjects, including age, weight, height, Fitzpatrick skin type, perfusion indices (PI), etc., are summarized in Table 1.

TABLE 1

| Variable. | | Female (N = 6) | | Male (N = 14) | |
|---|---|---|---|---|---|
| Demographics | Age (years) | 28.00 | (2.00) | 26.15 | (2.19) |
| | Fitzpatrick skin | 1.83 | (1.17) | 2.36 | (0.93) |
| | type (I-VI) | | | | |
| | Weight (kg) | 36.35 | (11.61) | 76.44 | (11.97) |
| | Height (cm) | 163.03 | (7.46) | 178.03 | (7.09) |
| | BMI (kg/m²) | 21.02 | (2.86) | 24.05 | (2.84) |
| Breath-hold | Baseline SpO₂ | 96.30 | (0.84) | 96.29 | (1.27) |
| | (%) | | | | |
| response | Nadir SpO₂ (%) | 88.80 | (4.81) | 80.32 | (8.70) |
| | Breath-hold | 44.07 | (25.64) | 55.99 | (15.32) |
| | duration (s) | | | | |
| | Approximate | 24.91 | (8.69) | 27.42 | (12.49) |
| | finger SpO₂ delay | | | | |
| | (s) | | | | |
| Perfusion index | Red PPG (%) | 0.05 | (0.03) | 0.07 | (0.04) |
| (PI) | Infrared PPG (%) | 0.08 | (0.04) | 0.10 | (0.06) |
| | Green PPG (%) | 0.56 | (0.23) | 0.60 | (0.40) |

Note values are presented in mean (standard deviation).

Table 1 provides demographics and physiological responses during the breath-hold of the subjects included in the analysis. Because the distribution of PI for red and infrared in the dataset falls well below the poor perfusion threshold (0.3%) as defined by the Food and Drug Administration (FDA) [16], the data suggests that this measurement site is malperfused.

In the breath-hold study, subjects were first asked to shave their chest hair to reduce interference. Subsequently, each subject performed 10 end-expiratory breath-holds while sitting in an upright posture with a one-minute break between breath-holds. One minute was found to be sufficiently long for SpO2 to return to its baseline level. Subjects were instructed to hold their breath for as long as possible. Throughout the study, subjects wore a nose clip and held the disposable mouthpiece (AFT36 bacteriological filter; Biopac System Inc, Santa Barbara, Calif) between their lips. After the data were collected, important oxygenation/deoxygenation events were manually labeled.

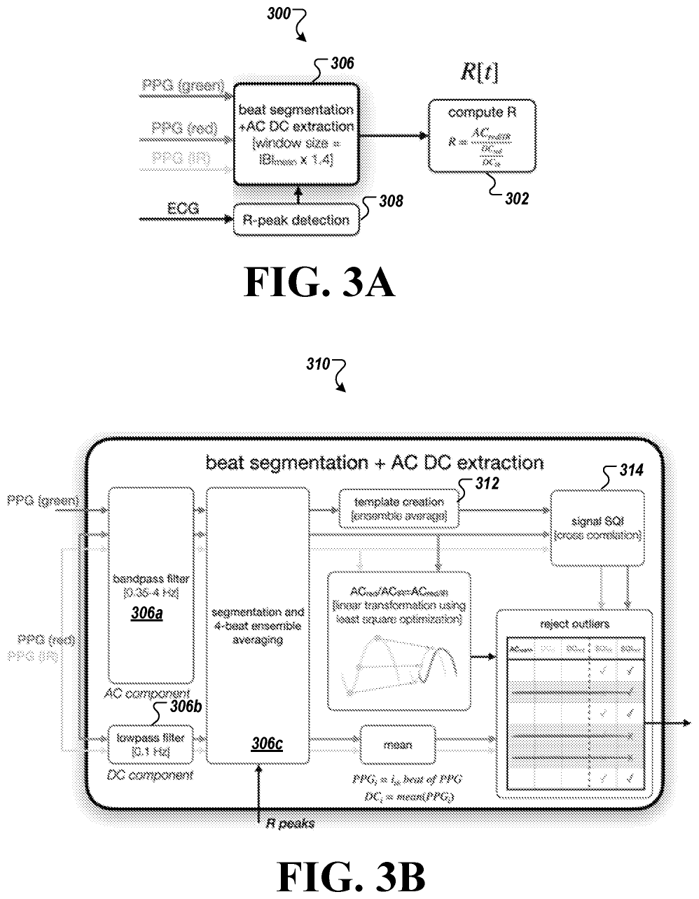

Referring now to FIG. 4, a schematic diagram 400 illustrating a subject 401 undergoing the breath hold study is provided. Placements of the wearable patch biosensor 403 are provided on the left side) and the ground truth Biopac sensors 405 are provided on the right side of the diagram 400. The relative size of the biosensor is shown with an off-the-shelf ECG electrode 407 and a penny 409. Note that the photodiode (PD) 420 has an area of 4.5 mm2. PD1 422 is on the left side, and PD2 424 is on the right side of the subject. As depicted in FIG. 4, the following information is collected: ECG (Biopac ECG100A; Biopac System Inc, Santa Barbara, CA, USA), right index finger SpO2 (Biopac OXY100E, TSD124A Finger Clip Transducer; Biopac System Inc), and respiratory flow (TSD117A Medium Flow Pneumotach Transducer; Biopac System Inc) data, all sampled at 2000 Hz. The Biopac OXY100E module reports an accuracy±2% for a SpO2 range of 70-100%. The 3M™ Red Dot™ ECG electrodes (model 2660; 3M, Saint Paul, MN, USA) was used throughout the study. Data outside of this SpO2 range were discarded since the accuracy is unknown.

In parallel, the wearable patch biosensor 403 was attached to the subject's mid-sternum and collected single-lead ECG, two sets of multiwavelength PPGs (red, infrared [IR], and green), and triaxial seismocardiogram (SCG) sampled at 500, 67, and 1000 Hz, respectively. The hardware used in the biosensor is almost identical to that reported in our previous work [8,10,23], except for the addition of the PPG modules and the change in form factor. The ECG analog front-end (AFE) and the accelerometer AFE (for SCG) remain the same. Specifically, the PPG AFE used to drive the LEDs and obtain data from the PDs is the Maxim 86170 (Maxim Integrated, San Jose, CA, USA). The multi-chip LEDs, which have red (660 nm), IR (950 nm), and green (526 nm) wavelengths, are the SFH 7016 (OSRAM, Munich, Germany), and the PDs are the VEMD 8080 (Vishay Semiconductors, Heilbronn, Baden-Württemberg, Germany). Serial Peripheral Interface was used as the communication protocol be-tween the microcontroller and peripheral sensors. This device is also equipped with wireless capabilities (i.e., Bluetooth and Wi-Fi) for transmitting data. However, in this study, data were stored in the Secure Digital card and later retrieved by a custom-built software application as in previous work [8, 10, 23]. The battery life of the device at the full sample rates of all sensors is up to 60 hours. The front and lateral views of the device are shown in FIG. 2A and FIG. 2B, discussed above.

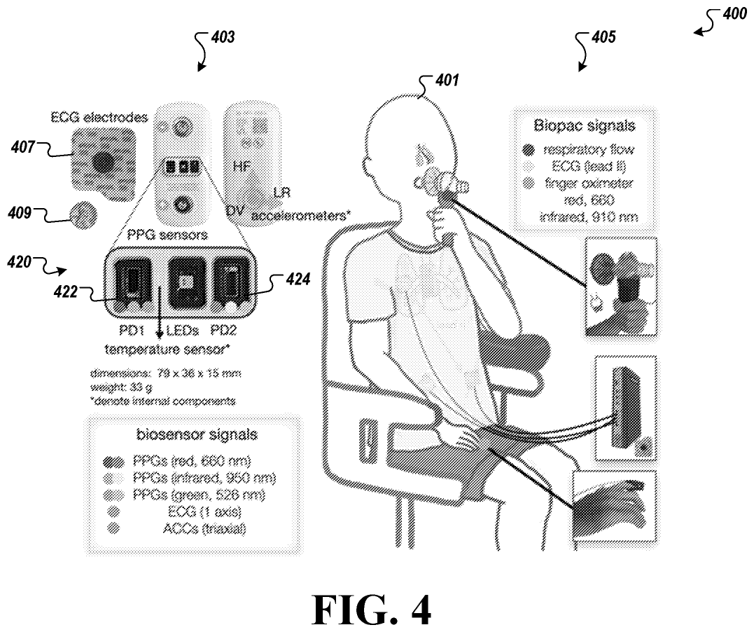

Example Acquired Signals. Referring now to FIG. 5, a schematic diagram 500 illustrating Biopac signals 501 and biosensor signals 503 during the fourth breath-hold 502 (labeled BH, blue) and during normal breathing 504 (pink) is provided. A 5 s window 510 during breath-hold is shown to illustrate the quality of the signals. As depicted in FIG. 5, filtered, high-quality physiological signals acquired during breath-hold and breathing are presented. For each subject, the photodiode (PD) with higher quality was selected as determined by visual inspection. The discrepancy can be attributed to the differences in LED/PD separation distance as LED/PD separation distance can affect the quality of PPG (p. 88). Further assessment may be needed since optimizing the LED/PD separation distance is a critical factor for obtaining a good quality signal. Manual labeling was performed using the respiratory flow and the SpO2 data. Alignment was necessary since it has been observed that deoxygenation events do not occur simultaneously for different body sites, and SpO₂ measured at the finger is usually delayed from SpO2 measured at central sites [4,5,24]. This delay can be partially attributed to the oxygen-conserving effect induced by breath-hold. Similar to the diving response [6], breath-hold also leads to bradycardia and peripheral vasoconstriction to reduce oxygen consumption in peripheries and redistribute blood flow to vital organs such as the brain and the heart [6]. The combined effect leads to a delayed deoxygenation measurement by a finger-pulse oximeter when compared to a pulse oximeter placed closer to the heart or the brain. Davies et al. reported a mean delay of 16.75±5.88 s across subjects for their in-ear reflectance pulse oximetry [4].

From the respiratory flow (top signal in FIG. 5), breathing 504 (the “oscillating” part, pink) and breath-hold 502 (the “silent” part, blue) segments can be easily distinguished. From the ground truth SpO₂ (the second signal in the Biopac signals 501 in FIG. 5), three distinct timestamps were recorded for each deoxygenation event: start, nadir, and end. The start of deoxygenation is defined as the point where SpO₂ begins to drop drastically (rate of SpO₂ decline>0.5%/cardiac cycle for 3 consecutive cardiac cycles). The nadir of deoxygenation is defined as the lowest SpO₂ within the deoxygenation event. The end of deoxygenation is defined as the point where SpO₂ returns to the baseline level. Usually, the nadir and the end of deoxygenation can be easily identified. To account for the delay of deoxygenation between finger and chest deoxygenation, the nadir deoxygenation of the finger is aligned to the end of the breath-hold, based on the assumption that chest arteries received well-oxygenated blood immediately following the end of the breath-hold. Though our results suggest this alignment procedure is somewhat accurate, it was found that a more precise alignment algorithm was required to achieve adequate accuracy.

Example Alignment Operation. The study determined the manually aligned R and SpO₂ samples were susceptible to delay due to a lack of ground truth SaO₂ data at the chest. To further reduce alignment error, a greedy algorithm may be used to find the delay that will minimize the alignment error. Specifically, all R and SpO₂^{rough}, which represent the SpO₂ roughly aligned using the manual label, may be used to train the parameters of the linear model described in Equation (10). Next, the trained linear model estimate SpO₂, [symbol] , from the samples of R may be used. An RMSE may be computed between SpO₂^{rough} and [symbol] of each breath-hold of each subject. Next, the RMSE between SpO₂^{rough, t}, which represents the SpO₂ roughly aligned using the manual label with a delay of t seconds and [symbol] may be evaluated. Finally, SpO₂^{precise}, SpO₂ more precisely aligned for each breath-hold of each subject may be found using Equation 7:

$$S p {O}_{2}^{precise} = ‖Sp {O}_{2} - {SpO}_{2}^{rough , t} {‖}_{2}^{2} \qquad ( Equation 7 )$$

The same optimization method may be repeated for all breath-holds of all subjects to complete the precise alignment procedure.

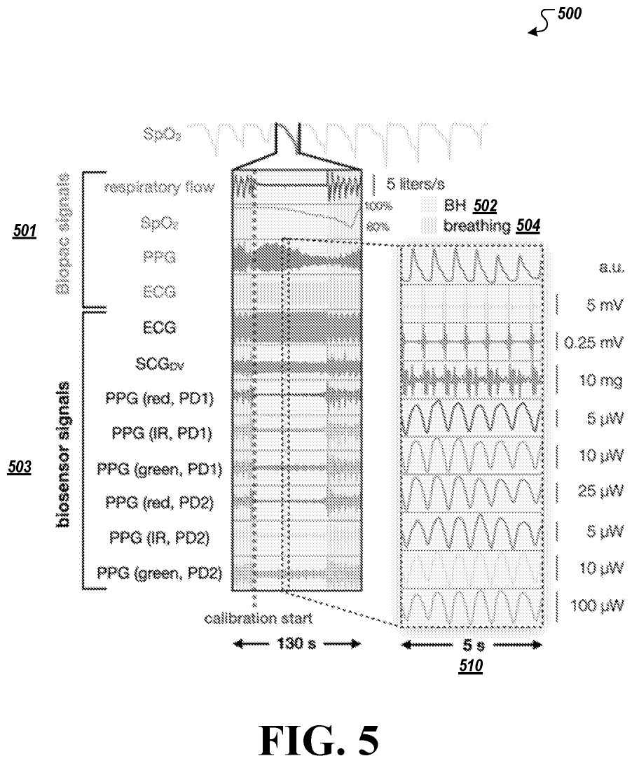

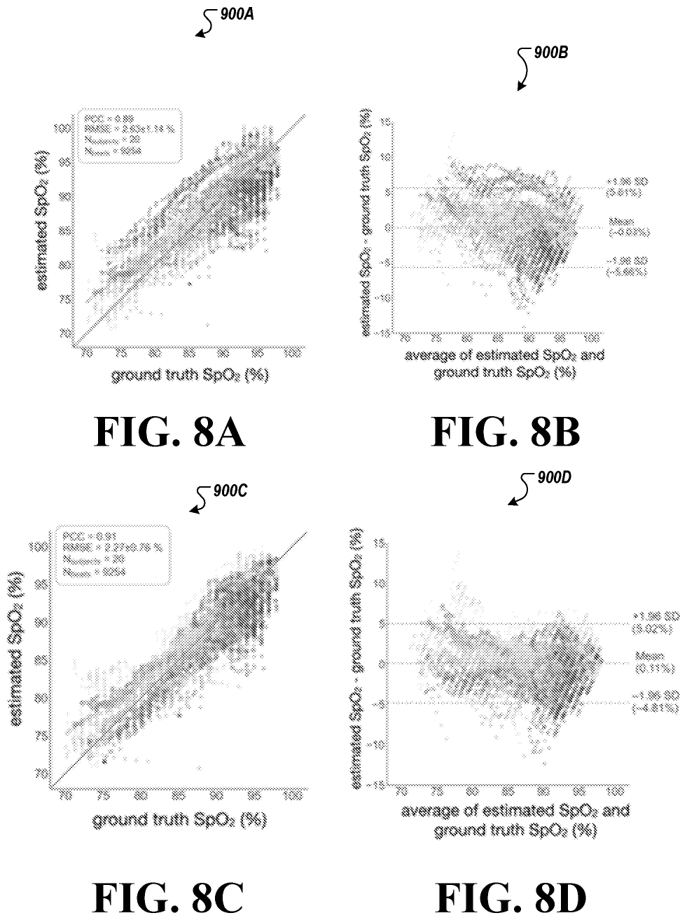

Example Preprocessing Overview. FIG. 6 is a schematic diagram 600 illustrating an example signal-preprocessing pipeline, which was used to extract R from the wearable patch biosensor signals. Although there are other signals in the dataset, it was found that six are particularly relevant to this work, namely, finger SpO₂, red PPG, IR PPG, green PPG, ECG, and respiratory flow. The Biopac signals 601 and biosensor signals 603 were first resampled to 500 Hz and synchronized by maximizing the cross-correlation of their ECG signals 605. The Biopac SpO₂ was further aligned to the biosensor signals on a per-breath-hold basis, using the manual label 607 described herein. Due to both respiratory artifacts when emerging from breath-hold and the lack of range in measured SpO₂ values, only target SpO₂ estimation was targeted during the breath-hold segments of the signals. Each extracted R from the breath-hold segment 609 was paired with the manually aligned SpO₂ 611, and both a scatter plot 610—demonstrating the correlation between R and SpO₂—and distributions plots to show the skewness of R and SpO₂—are depicted in FIGS. 9A-F. The skewness of R and SpO₂ can be partially attributed to our ability to maintain oxygenation homeostasis, enabled by the continuous supply of oxygen by the oxygen stores upon breath-hold [6]. Before uncovering the relationship between R and SpO₂, robust feature extraction and the outlier rejection algorithms necessary to extract R for the chest-based pulse oximetry are demonstrated. As depicted in FIG. 6, extracted pairs of the ratios (R) and SpO₂ were used for training and calibration of the model. T_{i, j} denotes the delay found between R and SpO₂ for the j^{th} breath-hold of subject i. The aligned data were displayed in the upper right scatter plot 610. The distribution of R and SpO₂ are also shown above and on the right of the scatter plot, respectively. IBIₘₑₐₙ denotes the mean of the interbeat intervals (IBI) of a subject.

Robust Feature Extraction via Linear Transformation. To compute R, it may be necessary to compute the AC features and the DC features of each PPG beat in certain embodiments. A feature may represent a scalar value to represent the characteristic of a PPG beat in the context of this work.

In FIG. 3B, the beat segmentation AC_{red/IR} extraction 306c, DC feature extraction 306c, and PPG_{green}-based outlier rejection (3e.g., 312, 314) are shown. In the study, to isolate the AC component, an empirically validated bandpass filter 306a, with a passband of 0.35 to 4 Hz, was first applied. The low cutoff was chosen to remove the baseline wander due to involuntary respiratory movement. The high cutoff was chosen empirically so as to reduce the dicrotic notch and preserve only the frequency components with less variation across wavelengths. The AC component was segmented into PPG beats using ECG R-peaks detected by a Pan-Tomkins algorithm [25], modified for R-peak correction, and further smoothed using 4-beat ensemble averaging. Conventionally, computing AC features for red and IR PPG relies on robust peak and valley extraction. Although respiratory artifacts were minimized through the breath-hold protocol, involuntary respiratory movements were still present and observable in some subjects. Evidently, extracting R robustly from respiration-corrupted PPG can be challenging [12,26]. Conventionally, the peak and valley of PPG in each cardiac cycle are extracted to compute the AC features (pp. 129-130). In a preliminary analysis, it was found that this method is not reliable as the signal can be easily distorted by the subtle—yet still significant—involuntary respiratory movements at this low perfusion site. To address this, a novel algorithm is introduced that does not require peak and valley extraction. Specifically, by rearranging the terms in Equation (1), the following is obtained using Equation 8:

$$R = \frac{A {C}_{red}}{A {C}_{I R}} / \frac{D {C}_{red}}{D {C}_{I R}} = {AC}_{r e d / I R} / \frac{D {C}_{red}}{D {C}_{I R}} \qquad ( Equation 8 )$$

By computing the AC_{red/IR}, the ratio of AC_{red} to AC_{IR}, the difficulty of extracting peaks and valleys in distorted PPG signals can be avoided. To do so, the fact that the IR PPG beat denoted as PPG_{IR}, appears to have a similar morphology to the red PPG beat, denoted as PPG_{red} is leveraged after being bandpass filtered. Therefore, the relationship of the two PPG beats can be modeled using a linear transformation method per Equation 9:

$${α}_{1}^{⋆} , {α}_{2}^{⋆} = ‖PP {G}_{r e d} - {α}_{1} P P {G}_{I R} - {α}_{2} {‖}_{2}^{2} \qquad ( Equation 9 )$$

In Equation 9, PPG_{red}, PPG_{IR} ∈ [symbol] ^{N}, N is the number of samples in the PPG beat, and α₁*, α₂* denote the scale and the bias that will minimize the [symbol] ²-norm of their differences. With the assumption that the differences, once optimized, should be closely distributed, beats with differences of more than five median absolute deviations from the median were rejected, which is a more robust rejection criterion compared to the “standard deviations around the mean” method [27]. Note that only 1.79% were rejected using this method. The optimal scale, α₁*, represents the ratio of the AC component of the two wavelengths per Equation 10.

$$A {C}_{r e d / I R} = {α}_{1}^{⋆} \qquad ( Equation 10 )$$

In parallel, the DC component was isolated using a low-pass filter (306b) with a high cutoff frequency of 0.1 Hz. This cutoff was based on a heuristic assumption that physiological dynamics of faster than 0.1 Hz (e.g., involuntary respiratory movement) do not directly relate to the deoxygenation induced by breath-hold based on data shown in [28-30]. The DC component was similarly segmented and smoothed (e.g., via 306c) to ensure consistency with the processing steps for AC extraction. Finally, DC features were computed as the mean of the segmented DC beats.

Evaluating Model Performance. To assess the performance of these three operations, the RMSE, the parameters of the linear model on a per-subject basis, and the Pearson correlation coefficient (PCC) of estimated SpO₂ on all subjects were jointly recorded. The mean and the standard deviation of the subject-specific RMSEs were computed to summarize the performance of each scheme and subsequently used as the critical metric to assess the capability of the pulse oximetry. Note that the errors presented in this work are all absolute errors rather than relative/percentage errors. The unit of RMSE is denoted by %, which represents the oxygen saturation level.

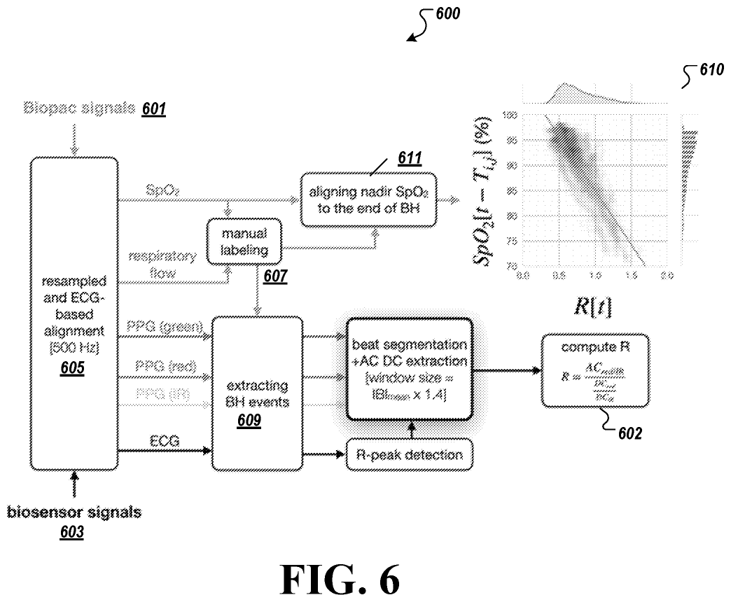

Computation of R. FIG. 7 shows different model training schemes 800, 802, and 804, respectively, in accordance with some illustrative embodiments.

The output matrix in FIG. 7 (802) has a dimension of N_{beats}×5, where the five columns represent the AC features (AC_{red/IR}), two DC features (DC_{red}, DC_{IR}), and the two binary SQI decisions (SQI_{red}, SQI_{IR}). Only features approved by the SQI algorithm were used to compute R. Note that the peak and valley methods were also experimented with, but it would require a more aggressive outlier rejection threshold (˜30% rejection ratio) to achieve comparable SpO₂ estimation accuracy. Finally, R is computed by dividing AC_{red/IR} by

$$\frac{D {C}_{red}}{D {C}_{I R}}$$

### per Equation 11.

$$R = A {C}_{r e d / I R} / \frac{D {C}_{red}}{D {C}_{I R}} = {α}_{1}^{⋆} / \frac{D {C}_{red}}{D {C}_{I R}} \qquad ( Equation 11 )$$

In this dataset, R is a unit-less measure and generally ranges from 0.4 to 1.6 for SpO₂ above 70%.

SpO₂ Estimation—Linear Regression. The temporally aligned SpO₂ and the extracted R were subsequently used to train the parameters in Equation 3. The parameters m (slope) and b (intercept) were estimated by minimizing the [symbol] ²-norm of the difference between the ground truth SpO₂ and estimated SpO₂ per Equation 12.

$${m}^{⋆} , {b}^{⋆} = {‖SpO}_{2} - m R - b {‖}_{2}^{2} = f ( x ) \qquad ( Equation 12 )$$

In Equation 12, x denotes pairs of SpO₂ and R, and f represents an arbitrary function for determining the optimal parameters of an objective function.

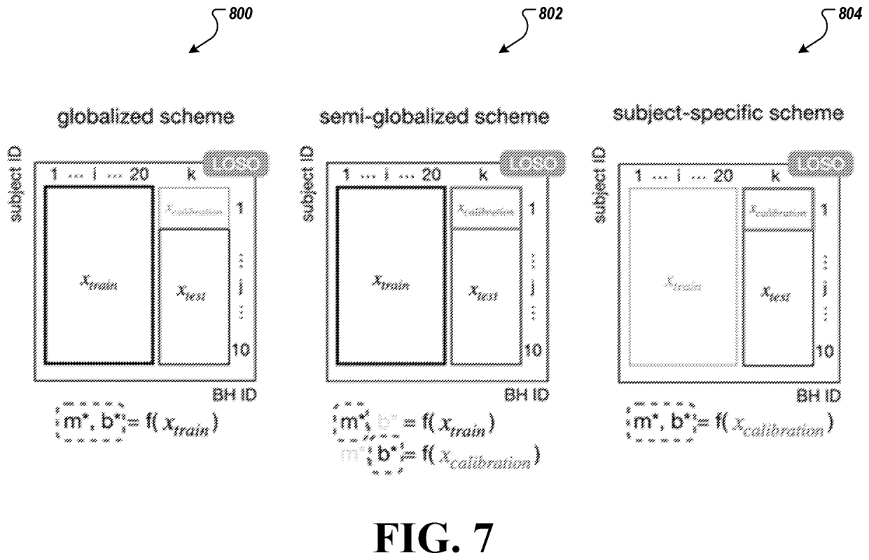

Training and Calibration Schemes. Since including a one-time, short calibration procedure is realistic for practical usage of the device, the best training and subject-specific calibration procedure were also investigated. Three training and calibration schemes were considered, including a (1) globalized scheme containing subject-independent training (see FIG. 8A); (2) semi-globalized scheme featuring global training with subject-specific calibration (see FIG. 8B); (3) subject-specific scheme (see FIG. 8C). The globalized scheme is equivalent to the standard LOSO cross validation. The semi-globalized scheme described herein is analogous to the semi-globalized method discussed in [33], aside from the fact that duration was used rather than number of points to standardize the subject-specific calibration. Particularly, in the semi-globalized scheme the globally trained intercept b was replaced by a subject-specific calibrated b (using the data in the first of the 10 breath-holds). The subject-specific scheme involved training both parameters using only the first breath-hold data of the subject. Calibration using m was also explored, but the results were considerably worse and therefore not reported. In both globalized and semi-globalized schemes, LOSO cross validation was also used to assess generalizability of the models trained. To compare model performance fairly and to avoid data leakage, the first breath-hold of the test subjects was excluded for evaluation of the globalized schemes to ensure identical testing data.

Results—Accuracy of SpO₂ estimation. In FIGS. 8A-F, regression plots and Bland-Altman plots are provided to demonstrate the estimation results. Table 2 shows a comparison of the results of three training schemes based on their RMSE, PCC, bias, and 95% LOR. The RMSEs across subjects, PCC, bias, and 95% limits of agreement (LOR) are summarized in Table 2.

TABLE 2

| | RMSE | PCC | bias | 95% LOR |
|---|---|---|---|---|
| Globalized | 2.63 ± 1.14% | 0.89 | −0.03% | [−5.66%, 5.61%] |
| Semi-globalized | 2.27 ± 0.76% | 0.91 | 0.11% | [−4.81%, 5.02%] |
| Subject-specific | 2.27 ± 0.88% | 0.92 | 0.24% | [−4.84%, 5.31%] |

The globalized scheme achieves lowest accuracy (see FIG. 8A and FIG. 8B). The semi-globalized scheme shows better accuracy (see FIG. 8C and FIG. 8D). The subject-specific scheme achieves the best accuracy (see FIG. 8E and FIG. 8F). Using the semi-globalized model, the mean RMSE was lowered by 0.36% and increase PCC by 0.02 when compared to the globalized model. The semi-globalized scheme and the subject-specific scheme have similar performance levels, both of which are superior to the globalized scheme. From the Bland-Altman plots, both models show minimal bias.

Semi-Globalized Scheme vs. Subject-Specific Scheme. Since it has not been previously examined in the literature, a study of which parameters benefit the most from subject-specific calibration was conducted. This is accomplished by comparing the semi-globalized scheme (i.e., calibrating b) to the subject-specific scheme (i.e., calibrating both m and b) while varying the calibration duration constraints.

FIG. 9 is a graph showing the results of different calibration methods. In particular, FIG. 9 shows calibration using b and using both b and m. ns denotes not significant (p>0.05), and a double asterisk denotes significant differences (p<0.01) as determined by paired sample t-tests.

The duration constraint was imposed by considering data only within the said duration. Surprisingly, the semi-globalized model works more efficiently at reducing RMSE, as shown in FIG. 9. In FIG. 9, outlier subjects were excluded for better visualization. In all three duration constraints (10 s, 20 s, and 30 s), the semi-globalized schemes achieved a lower RMSE. When comparing the RMSE of calibrating b, constrained by a calibration duration of 10 s, to calibrating both parameters by a calibration duration of 30 s, no statistical significance (p>0.05) was found as determined by a paired sample t-test. Therefore, it was determined that the semi-globalized scheme is the best calibration strategy for this dataset as it would require a shorter duration to achieve similar performance to the subject-specific scheme.

Standardizing Subject—Specific Calibration: Duration vs. SpO₂ Range. To study the most efficient way to collect data for calibration, the changes in RMSE were examined by imposing different constraints on the calibration data, including a duration constraint and SpO₂ range constraint. Similar to the duration constraint, the SpO₂ range constraint considers data only within the said SpO₂ range.

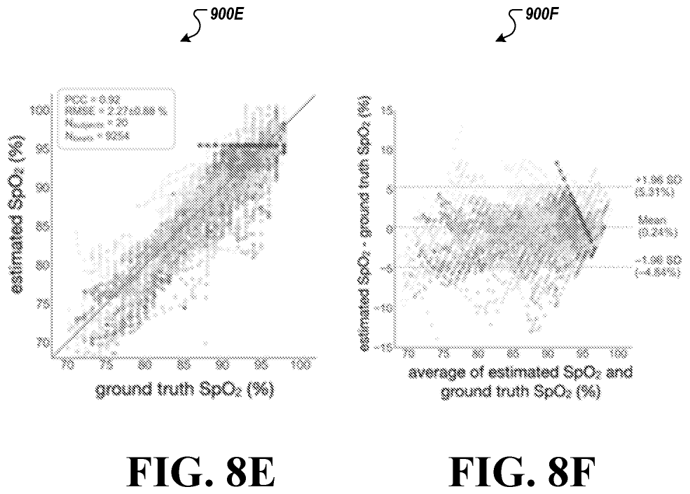

FIGS. 10A-10B are graphs 1200A, 1200B, respectively, showing the results of calibration analysis. According to the results shown in FIG. 10A, increasing calibration duration from 1 s to 20 s while fixing the SpO₂ range to 30% leads to a significant (p<0.05) reduction in mean RMSE across the subjects. On the other hand, the results shown in FIG. 10B suggest that increasing the calibration SpO₂ range from 1% to 20% while fixing the duration to 30 s did not lead to a significant (p>0.05) difference in mean RMSE. Paired sample t-tests were used for the statistical analysis. Standardizing calibration duration appears to be the best calibration strategy here.

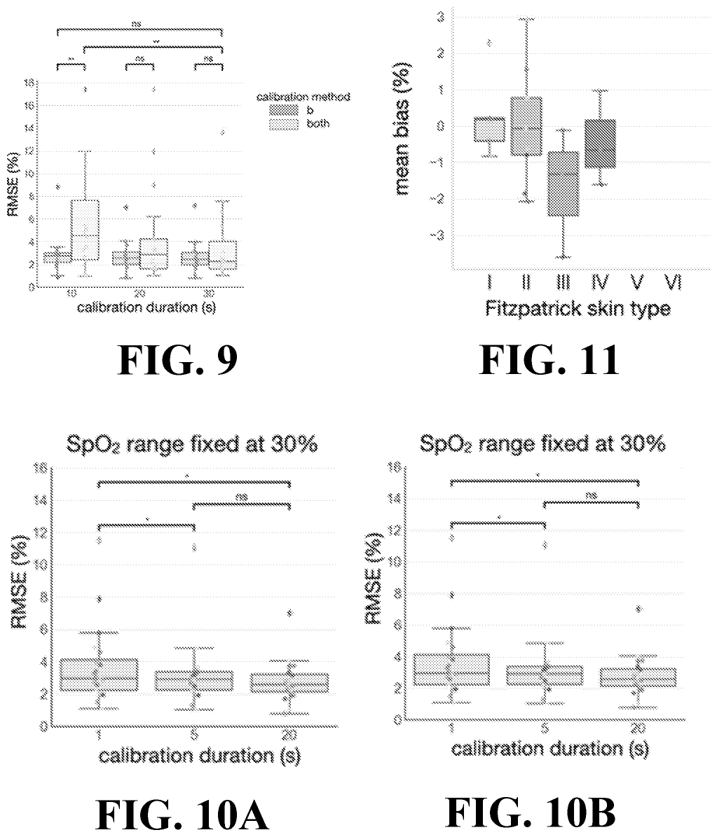

Effect of Varying Melanin Content. Since none of the subjects had nail polish on their right index finger or tattoos on their sternum, only the confounding effect of the difference in melanin content was considered. In this analysis, melanin levels were assessed using self-reported Fitzpatrick skin types and studied the way melanin content affects the bias between finger and sternum SpO₂. FIG. 11 is a graph showing Mean bias across varying Fitzpatrick skin types. In FIG. 11, the errors between different Fitzpatrick skin types are shown to be statistically insignificant (p>0.05), using a one-way analysis of variance (ANOVA). This implies that our device does not introduce different bias for subjects with varying skin melanin content when compared to the finger pulse oximeter, understandably, as both operate on the same principles. However, it is worth noting that there are five subjects for skin type I, nine subjects for skin type II, three subjects for skin type III, three subjects for skin type IV, and zero subjects for skin types V and VI. Due to the limited sample size and lack of data for the darkest Fitzpatrick skin types, the results attained here may not provide meaningful insight into the true accuracy of the proposed chest-based pulse oximetry on persons of all melanin levels. Note that in [35,36], it was reported that melanin content leads to SpO₂ overestimation at low SaO₂. Further investigation is required to study the way melanin content affects the accuracy of chest-based pulse oximetry at various SaO₂ levels.

Previous evaluations of central-pulse oximetry and addressed relevant concerns were unified while showing an accuracy that is comparable to the state-of-the-art [13]. To the best of our knowledge, this is the first thorough evaluation of chest-based pulse oximetry that jointly features a sufficient sample size, a wide dynamic range of SpO₂, minimal respiratory artifacts, and rigorous cross-validation to avoid data leakage. Furthermore, the study protocol, our alignment method, and key algorithmic components were described in full detail to allow for replication. This work paves the way for realizing the simultaneous monitoring of, in addition to SpO₂, the cardiac, pulmonary, and cardiopulmonary functions using a small, standalone wearable patch device continuously and remotely, unlocking opportunities in personalized health intervention outside of a clinical setting.

Accurate SpO₂ Estimation. Low mean RMSEs were achieved for all training and calibration schemes, which were well within the criteria (RMSE≤3.5%) for reflective pulse oximetry outlined by the FDA standard [16]. The instant study addressed the challenges of the aforementioned approaches and estimated SpO₂ accurately using a novel algorithm that proves to be robust for PPG measured at this poorly perfused site. The breath-hold protocol success-fully induced hypoxemia and reduced respiratory artifacts. Furthermore, the novel algorithms described herein to derive R leverage the morphological similarity between PPG_{red} and PPG_{IR}. Our method avoids peak and valley extraction for distorted PPG beat and proves to be less susceptible to artifacts. The PPG_{green}-based outlier rejection algorithm was inspired by the robustness of PPG_{green} against motion artifacts [31]. Together, they alleviated difficulty in feature extraction for most PPG beats and excluded undesired PPG beats robustly.

Standardization of Subject—Specific Calibration. Besides accurate SpO₂ measurements, experiments were designed to identify the best training and calibration for this dataset for improving RMSE. More data points help to better calibrate the model to the test subject, and they do so by reducing the noise in the R extracted rather than capturing a wider SpO₂ dynamic range, as evident from the results in FIGS. 8A-F. Subject-specific calibration of b helps to reduce the randomness in the data. In contrast, if m alone were calibrated, the SpO₂ range is expected to have a more important role. Ultimately, both longer calibration duration and wider SpO₂ range may be beneficial, but in a situation where hypoxemia is not preferred by the intended users, the present disclosure shows that a limited SpO₂ dynamic range with a sufficiently long calibration duration can still efficiently improve accuracy. This finding is beneficial for the usage of the device since it alleviates the need to induce changes in SpO₂ and consequently makes the subject-specific calibrating procedure more practical and safer. Consequently, if necessary, breath-hold duration should be standardized instead of the SpO₂ range.

When observing the data used to calibrate both m and b for test subjects, the first breath-hold was consistently shorter across subjects. As a direct consequence, data of the first breath-hold generally does not have a wide SpO₂ dynamic range. Furthermore, subjects seemed to be able to hold their breath longer due their adaptive tolerance to withstand the vaguely defined “discomfort” [37]. Since estimating the slope m requires a sufficient dynamic range of SpO₂, calibrating m using just the first breath-hold is usually not enough. Using all 10 breath-holds and adequate SpO₂ dynamic range across all training subjects offers a clear advantage to the globally trained m over the calibrated m. Hence, when the calibration data were limited (within the duration of one breath-hold), training m globally and calibrating b using the test subject's data can achieve better accuracy.

Example Clinical Use Case. The current manufacturing cost of the device is on the order of $200. However, producing this device on a large scale can reduce the cost substantially as components would be ordered in volume and manufacturing processes can be refined to improve manufacturability. Challenges with scalability are not anticipated as the devices manufactured so far show robust functionality. The practical subject-specific calibration procedure can be designed by aggregating the conclusions made thus far. A 15 s breath-hold is suggested during which the ground truth SpO₂ and biosensor data are collected from a target subject. This breath-hold duration is selected because all breath-hold durations across the subjects in this study exceed 15 s. Using the data from the subjects analyzed in this study, the globally trained slope m_{global} was found to be −21.54 and the intercept b_{global} was found to be 106.69. Following the semi-globalized scheme, b_{global} can be replaced by b_{subject-specific}, which was calibrated using data from the 15 s breath-hold of the target subject. The resulting subject-specific linear model takes the form of SpO₂=m_{global}×R+b_{subject-specific}. One potential reason for calibration failure could be the adoption of smoking behavior. According to [38], smokers have elevated levels of carboxyhemoglobin (COHb). As a result, assumptions (i.e., the only hemoglobin species in the arteries are Hb and HbO₂) made in Equation (2) are violated, which can subsequently lead to the overestimation of SpO₂.

The exemplary system and method may be employed as described in applications that can withstand respiratory artifacts more severe than the involuntary respiratory movements during breath-hold and attain similar accuracy.

In some embodiments, the exemplary system and method may be used to better inform underlying pulmonary dysfunctions unobtrusively, continuously, and remotely. Together, with its ability to measure cardiac function, the wearable patch biosensor can be validated for its ability to quantitatively and objectively assess disease progression of cardiovascular and pulmonary diseases such as COVID-19, nocturnal hypoxia caused by sleep apnea, and high-altitude sickness. Ultimately, tracking these health parameters may provide a better understanding of the cardiopulmonary-related comorbidities and consequently facilitate the adoption of longitudinal wearable monitoring devices, for detecting underlying disease when symptoms are subtle and unnoticeable.

It should be appreciated that the logical operations described above and in the appendix can be implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

Machine Learning. In addition to the machine learning features described above, the various analysis system can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an ANN is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

### Example Computing System

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device 200 to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory 230, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory 230, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### Discussion

In some embodiments, a chest-worn wearable patch biosensor is disclosed capable of accurately estimating SpO₂ while subjects underwent a 10 breath-hold protocol. By standardizing the calibration duration, rather than calibration range, optimal calibration can be achieved. This innovation is described in our recent publication by Chan et al. [40]. PPG beats may be distorted due to motion artifacts and involuntary respiratory movements and therefore can hinder accurate SpO₂ estimation. Hence, a novel outlier rejection algorithm is provided using the green PPG beats as a signal quality template for its robustness against noise [31], so as to reduce the contamination of abnormal features extracted. Our signal quality assessment relied on two assumptions: (1) reliable red or IR PPG beats in the bandwidth filtered constitute a morphology similar to that of green PPG beats; and (2) outliers in AC ratios are defined as datapoints that deviate by more than five median absolute deviations from the median. To determine the similarity, a methodology described in is considered. First, the normalized cross-correlation (NCC) between a PPG beat with its corresponding template is computed. Since each PPG (&lambda;) is reflective of different tissue composition, there may exist a slight delay among them. Note &lambda; represents the wavelength of the PPG beat. To account for this delay, NCC was computed between the template PPG beat and the test PPG beat with different lags. The maximal NCC was selected as the SQI of PPG (&lambda;), which has a range of [−1, 1]. While outlier rejection algorithms exist, none utilizes the green PPG with NCC to construct an SQI measure [17]. This innovation allows for leveraging the easy incorporation of green PPG to commercially available pulse oximeter to improve their accuracy when motion artifacts are present.

Conventional technologies rely on analyzing the morphologies of red and infrared PPG (e.g., largest slope, presence of dicrotic notch, etc.) and comparing them with past data [1]. However, there are a few obvious shortcomings. First, if the past data used for comparison are distorted, the accepted red and infrared PPG data may also be distorted. Second, if the rejection algorithm is too sensitive, the rejected data may be wasted. Since this invention does not use past data of the red and infrared PPG, this invention has the advantages of rejecting corrupted PPG data more accurately, owning to the reliable template extracted from the green PPG. [1] King, P. H. Design Of Pulse Oximeters. IEEE Engineering in Medicine and Biology Magazine 1998, 17, 117&ndash; 117.

Examples of these PPG system are provided below.
1. WO 2017/089921 A1 METHOD TO QUANTIFY PHOTOPLETHYSMOGRAM (PPG) SIGNAL QUALITY.
2. U.S. Pat. No. 7,024,233 B2 PULSE OXIMETRY DATA CONFIDENCE INDICATOR.
3. U.S. Pat. No. 7,890,154 B2 SELECTION OF ENSEMBLEAVERAGING WEIGHTS FOR A PULSE OXMETER BASED ONSIGNAL QUALITY METRICS.
4. U.S. Pat. No. 9,351,674 B2 METHOD FOR ENHANCING PULSE OXMETRY CALCULATIONS IN THE PRESENCE OF CORRELATED ARTIFACTS.
5. U.S. Pat. No. 6,987,994 B1 PULSE OXIMETRY SPO2 DETERMINATION.

In contrast, the exemplary system and method address the poor green PPG quality severely corrupted by respiratory artifacts. If the green PPG is corrupted, the template constructed will not be representative of the true pulsatile dynamics of the underlying tissue. As a result, the outlier rejection algorithm may not be accurate. Improvement can be employed using, for example, an adaptive filter that uses respiratory artifacts as a reference while collecting data necessary for validating this improved invention.

More validated experiments are required to demonstrate its safety and efficacy for FDA approval. The described method is implemented and tested on offline data. Improvements may be added when the system is tested on online data.

It is noted that due to the COVID-19 pandemic, the need for monitoring SpO₂ in an unobtrusive manner has substantially increased. Currently, there is a rising awareness of pulse oximeters used for COVID-19 management as they can be used to optimize the usage of medical resources, to implement timely intervention strategies, and to monitor post-COVID conditions. The current 7-day moving average of daily new cases (122,297) consists of 0.037% of the U.S. population [1]. Additionally, WHO guidance states that pulse oximeters should be considered in symptomatic patients with COVID-19 and risk factors for progression to severe disease who are not hospitalized for home monitoring [2].

Due to the novel Coronavirus disease (COVID-19) pandemic, there is a clear need to monitor respiratory functions in outpatient settings to help assess the progression of COVID-19 during the presymptomatic, symptomatic, and recovery stages. In a recent effort to record and model the trajectories of several vital signs in hospitalized COVID-19 patients, Pimentel et al. showed that peripheral oxygen saturation (SpO₂) is among the most indicative of parameters of COVID-19 progression prior to primary outcomes, suggesting the importance to monitor SpO₂ continuously [1]. Through remote SpO₂ monitoring, accurate tracking of COVID-19 progression allows for the implementation of disease-management strategies for both timely interventions and the optimization of scarce medical resources [2].

Unfortunately, existing SpO₂ measurement devices are inconvenient for monitoring in outpatient settings. Typically, SpO₂ is measured through pulse oximeters placed at peripheral extremities such as the fingers; however, these devices obstruct normal activities of daily living (ADLs) due to restriction of finger usage. In addition, finger-clip-based pulse oximeters are accordingly limited in practice to intermittent or single-point measurements. Recently, commercial wrist-worn devices such as Apple Watch, Fitbit Sense Advanced Smartwatch, and Garmin Vivosmart have been developed that allow for more convenient monitoring and offer continuous SpO₂ measurements. Unfortunately, SpO₂ measured at the wrist is likely more susceptible to motion artifacts when compared to measurement sites closer to the center of mass of the body, such as the forehead and ear during walking, as shown by Longmore et al. [3]. In addition, peripheral sites such as the wrist respond to apnea events at a slower than central sites [4,5] due to the redistribution of blood flow, oxygen conservation [6], and their distal location to the heart.

Thus, central pulse oximeters offer a promising approach for ambulatory outpatient monitoring and detecting acute hypoxemia events. Furthermore, chest-based pulse oximeters might be advantageous for their synergistic incorporation with other cardiac monitoring methods—such as electrocardiogramd seismocardiographic—for a more holistic understanding of cardiac functions [7-10]. Nevertheless, a few major challenges, such as limitations to the reflectance design, the presence of respiratory artifacts, and the malperfusion of the sternum, pose difficulties to the adoption of chest-based approaches. Despite these challenges, chest-based approach may be feasible [3,11-15].

However, existing methods using chest-based devices are not rigorously validated, and thus more work is needed to advance chest-worn pulse oximetry. Specifically, validating chest-based pulse oximetry for continuous monitoring would require a sufficiently large and diverse subject population, a wide dynamic range of SpO₂, a resultant root-mean-square error (RMSE) lower than 3.5% [16], and ideally, a form factor that does not interfere with ADLs. Upon investigation, there are some clear gaps in the existing literature in these areas that should be addressed. For example, either insufficient sample size (N=1) in or narrow dynamic range for SpO₂ (88-99%) in limits the validity of the accuracy of their approaches. Meanwhile, Näslund et al. showed a strong agreement between their estimated SpO₂ and arterial oxygen saturation (SaO₂); however, their device was unable to capture the pulsatile component of the PPG signals and, therefore might be susceptible to motion artifacts and skin pigmentation (p. 34). Kramer et al. achieved accurate SpO₂ estimations (RMSE of 2.9%, N=13), but their work lacks the key details necessary for replicating their algorithm, such as preprocessing and feature extraction. Finally, Vetter et al. conducted a study following the International Organization for Standardization 9919 international standard, included a moderate number of subjects (N=10) and a wide dynamic range of SpO₂ (70-99%), and provided sufficient details of their approach. Nevertheless, the prototype appears to be cumbersome to use as it requires a chest strap to affix the device for sufficient contact pressure. Additionally, due to the lack of leave-one-subject-out (LOSO) cross-validation for training and testing the model, their method is susceptible to data leakage, and therefore the high accuracy attained may not be generalizable. To the best of our knowledge, there exists no known accurate chest-based pulse oximetry approach that has been thoroughly validated within the literature. This work attempts to address these shortcomings and present a chest-based pulse oximeter that can estimate SpO₂ in close agreement with a commercially available, validated finger pulse oximeter.

The instant study demonstrates the feasibility of our small, standalone chest-based wearable patch biosensor. In this work, data was collected from 20 subjects who underwent a breath-hold perturbation to induce hypoxia and used PPG signals from a custom chest-based biosensor to estimate SpO₂. To estimate SpO₂ robustly, an algorithm is presented to extract key PPG features to account for poor perfusion at the sternum and involuntary respiratory artifacts [19]. In addition, a PPG_{green}-based outlier rejection algorithm was developed for rejecting red and infrared (IR) PPG beats of lower quality. Finally, the optimal calibration scheme for practical usage of this chest-based pulse oximetry was demonstrated. These operations provide for continuous monitoring of SpO₂.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Moreover, the various components may be in communication via wireless and/or hardware or other desirable and available communication means, systems and hardware. Moreover, various components and modules may be substituted with other modules or components that provide similar functions.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

The processing unit may be a standard programmable processor that performs arithmetic and logic operations necessary for the operation of the computing device. Multiple processors may be employed. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

It should be appreciated that the logical operations described above can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include the one particular value and/or the other particular value.

By “comprising” or “containing” or “including” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

It should be appreciated that, as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
[1] Pimentel, M. A. F.; Redfern, O. C.; Hatch, R.; Young, J. D.; Tarassenko, L.; Watkinson, P. J. Trajectories of Vital Signs in Patients with COVID-19. Resuscitation 2020, 156, 99-106, doi: 10.1016/j.resuscitation.2020.09.002.
[2] Shah, S.; Majmudar, K.; Stein, A.; Gupta, N.; Suppes, S.; Karamanis, M.; Capannari, J.; Sethi, S.; Patte, C. Novel Use of Home Pulse Oximetry Monitoring in COVID-19 Patients Discharged from the Emergency Department Identifies Need for Hospitalization. Acad. Emerg. Med. 2020, 27, 681-692, doi: 10.1111/acem.14053.
[3] Longmore, S. K.; Lui, G. Y.; Naik, G.; Breen, P. P.; Jalaludin, B.; Gargiulo, G. D. A Comparison of Reflective Photoplethysmography for Detection of Heart Rate, Blood Oxygen Saturation, and Respiration Rate at Various Anatomical Locations. Sensors 2019, 19, 1874, doi: 10.3390/s19081874.
[4] Davies, H. J.; Williams, I.; Peters, N. S.; Mandic, D. P. In-Ear SpO₂: A Tool for Wearable, Unobtrusive Monitoring of Core Blood Oxygen Saturation. Sensors 2020, 20, 4879, doi: 10.3390/s20174879.
[5] Sugino, S.; Kanaya, N.; Mizuuchi, M.; Nakayama, M.; Namiki, A. Forehead Is as Sensitive as Finger Pulse Oximetry during General Anesthesia. Can. J. Anesth. 2004, 51, 432-436, doi: 10.1007/BF03018304.
[6] Bouten, J.; Bourgois, J. G.; Boone, J. Hold Your Breath: Peripheral and Cerebral Oxygenation during Dry Static Apnea. Eur. J. Appl. Physiol. 2020, 120, 2213-2222, doi: 10.1007/s00421-020-04445-y.
[7] Inan, O. T.; Pouyan, M. B.; Javaid, A. Q.; Dowling, S.; Etemadi, M.; Dorier, A.; Heller, J. A.; Bicen, A. O.; Roy, S.; De Marco, T.; et al. Novel Wearable Seismocardiography and Machine Learning Algorithms Can Assess Clinical Status of Heart Failure Patients. Circ-Heart. Fail. 2018, 11, e004313, doi: 10.1161/CIRCHEARTFAILURE.117.004313.
[8] Semiz, B.; Carek, A. M.; Johnson, J. C.; Ahmad, S.; Heller, J. A.; Garcia-Vicente, F. G.; Caron, S.; Hogue, C. W.; Etemadi, M.; Inan, O. Non-Invasive Wearable Patch Utilizing Seismocardiography for Peri-Operative Use in Surgical Patients. IEEE J. Biomed. Health Inform. 2021, 25, 1572-1582, doi: 10.1109/JBHI.2020.3032938.
[9] Aydemir, V. B.; Nagesh, S.; Shandhi, M. M. H.; Fan, J.; Klein, L.; Etemadi, M.; Heller, J. A.; Inan, O. T.; Rehg, J. M. Classification of Decompensated Heart Failure From Clinical and Home Ballistocardiography. IEEE Trans. Bio-Med. Eng. 2020, 67, 1303-1313, doi: 10.1109/TBME.2019.2935619.
[10] Shandhi, M. M. H.; Bartlett, W. H.; Heller, J. A.; Etemadi, M.; Young, A.; Plötz, T.; Inan, O. T. Estimation of Instantaneous Oxygen Uptake During Exercise and Daily Activities Using a Wearable Cardio-Electromechanical and Environmental Sensor. IEEE Trans. Inf. Technol. B. 2021, 25, 634-646, doi: 10.1109/JBHI.2020.3009903.
[11] Schreiner, C.; Catherwood, P.; Anderson, J.; Mclaughlin, J. Blood Oxygen Level Measurement with a Chest-Based Pulse Oximetry Prototype System. In Proceedings of the Computing in Cardiology, Belfast, UK, 26-29 Sep. 2010; pp. 537-540.
[12] AKiruthiga, A.; Alex, A.; Balamugesh, T.; Prabhu, D.; Christopher, D. J.; Preejith, S. P.; Joseph, J.; Sivaprakasam, M. Reflectance Pulse Oximetry for Blood Oxygen Saturation Measurement from Diverse Locations-A Preliminary Analysis. In Proceedings of the 2018 IEEE International Symposium on Medical Measurements and Applications (MeMeA), Rome, Italy, 11-13 Jun. 2018; pp. 1-6.
[13] Kramer, M.; Lobbestael, A.; Barten, E.; Eian, J.; Rausch, G. Wearable Pulse Oximetry Measurements on the Torso, Arms, and Legs: A Proof of Concept. Mil. Med. 2017, 182, 92-98, doi: 10.7205/MILMED-D-16-00129.
[14] Vetter, R.; Rossini, L.; Ridolfi, A.; Sola, J.; Chetelat, O.; Correvon, M.; Krauss, J. Frequency Domain SpO₂ Estimation Based on Multichannel Photoplethysmographic Measurements at the Sternum. In Proceedings of the World Congress on Medical Physics and Biomedical Engineering, Munich, Germany, 7-12 Sep. 2009; pp. 326-329, ISBN 978-3-642-03881-5.
[15] Näslund, E.; Lindberg, L.-G.; Lund, I.; Näslund-Koch, L.; Larsson, A.; Frithiof, R. Measuring Arterial Oxygen Saturation from an Intraosseous Photoplethysmographic Signal Derived from the Sternum. J. Clin. Monit. Comput. 2020, 34, 55-62, doi: 10.1007/s10877-019-00289-w.
[106] Pulse Oximeters—Premarket Notification Submissions [510 (k)s] Guidance for Industry and Food and Drug Administration Staff. Available online: https://www.fda.gov/regulatory-information/search-fda-guidance-documents/pulse-oximeters-premarket-notification-submissions-510ks-guidance-industry-and-food-and-drug (accessed on 12 Dec. 2021).
[17] King, P. H. Design Of Pulse Oximeters. IEEE Eng. Med. Biol. 1998, 17, 117-117.
[18] Arnold, M. The Surgical Anatomy of Sternal Blood Supply. J. Thorac. Cardiovasc. Surg. 1972, 64, 596-610, doi: 10.1016/S0022-5223 (19) 39718-1.
[19] Andersson, J.; Schagatay, E. Effects of Lung Volume and Involuntary Breathing Movements on the Human Diving Response. Eur. J. Appl. Physiol. 1997, 77, 19-24, doi: 10.1007/s004210050294.
[20] Reisner, A.; Shaltis, P. A.; McCombie, D.; Asada, H. H.; Warner, D. S.; Warner, M. A. Utility of the Photoplethysmogram in Circulatory Monitoring. Anesthesiology 2008, 108, 950-958, doi: 10.1097/ALN.0b013e31816c89e1.
[21] Vijayarangan, S.; Suresh, P.; SP, P.; Joseph, J.; Sivaprakasam, M. Proceedings of Robust Modelling of Reflectance Pulse Oximetry for SpO₂ Estimation. In Proceedings of the 2020 42nd Annual International Conference of the IEEE Engineering in Medicine Biology Society (EMBC), Montreal, QC, Canada, 20-24 Jul. 2020; pp. 374-377.
[22] Priem, G.; Martinez, C.; Bodinier, Q.; Carrault, G. Clinical Grade SpO₂ Prediction through Semi-Supervised Learning. In Proceedings of the 2020 IEEE 20th International Conference on Bioinformatics and Bioengineering (BIBE), Cincinnati, OH, USA, 26-28 Oct. 2020; pp. 914-921.
[23] Ganti, V.; Carek, A. M.; Jung, H.; Srivatsa, A. V.; Cherry, D.; Johnson, L. N.; Inan, O. T. Enabling Wearable Pulse Transit Time-Based Blood Pressure Estimation for Medically Underserved Areas and Health Equity: Comprehensive Evaluation Study. JMIR mHealth and uHealth 2021, 9, e27466, doi: 10.2196/27466.
[24] Reynolds, L. M.; Nicolson, S. C.; Steven, J. M.; Escobar, A.; McGonigle, M. E.; Jobes, D. R. Influence of Sensor Site Location on Pulse Oximetry Kinetics in Children. Anesth. Analg. 1993, 76, 751-754, doi: 10.1213/00000539-199304000-00011.
[25] Pan, J.; Tompkins, W. J. A Real-Time QRS Detection Algorithm. IEEE Trans. Biomed. Eng. 1985, 32, 230-236, doi: 10.1109/TBME.1985.325532.
[26] Tanveejul, A. P. M.; Alex, A.; Preejith, S. P.; Christopher, D. J.; Chandy, S.; Joseph, J.; Sivaprakasam, M. A Study on the Subject and Location Specificity in Reflectance Based SpO₂ Estimation Using R-Value Based Calibration Curve. In Proceedings of the 2020 IEEE International Symposium on Medical Measurements and Applications (MeMeA), Bari, Italy, 1 June-1 Jul. 2020; pp. 1-6.
[27] Leys, C.; Ley, C.; Klein, O.; Bernard, P.; Licata, L. Detecting Outliers: Do Not Use Standard Deviation around the Mean, Use Absolute Deviation around the Median. J. Exp. Soc. Psychol. 2013, 49, 764-766, doi: 10.1016/j.jesp.2013.03.013.
[28] Andersson, J. P. A.; Liner, M. H.; Rünow, E.; Schagatay, E. K. A. Diving Response and Arterial Oxygen Saturation during Apnea and Exercise in Breath-Hold Divers. J. Appl. Physiol. 2002, 93, 882-886, doi: 10.1152/japplphysiol.00863.2001.
[29] Andersson, J.; Schagatay, E. Arterial Oxygen Desaturation during Apnea in Humans. Undersea Hyperb. Med. 1998, 25, 21-25.
[30] Stewart, I. B.; Bulmer, A. C.; Sharman, J. E.; Ridgway, L. Arterial Oxygen Desaturation Kinetics during Apnea. Med. Sci. Sports Exerc. 2005, 37, 1871-1876, doi: 10.1249/01.mss.0000176305.51360.7e.
[31] Matsumura, K.; Toda, S.; Kato, Y. RGB and Near-Infrared Light Reflectance/Transmittance Photoplethysmography for Measuring Heart Rate During Motion. IEEE Access 2020, 8, 80233-80242, doi: 10.1109/ACCESS.2020.2990438.
[32] Krasteva, V.; Jekova, I. QRS Template Matching for Recognition of Ventricular Ectopic Beats. Ann. Biomed. Eng. 2007, 35, 2065-2076, doi: 10.1007/s10439-007-9368-9.
[33] Ganti, V. G.; Carek, A.; Nevius, B. N.; Heller, J.; Etemadi, M.; Inan, O. Wearable Cuff-Less Blood Pressure Estimation at Home via Pulse Transit Time. IEEE J. Biomed. Health Inform. 2021, 25, 1926-1937, doi: 10.1109/JBHI.2020.3021532.
[34] Fitzpatrick, T. B. The Validity and Practicality of Sun-Reactive Skin Types I Through VI. Arch. Dermatol. 1988, 124, 869-871, doi: 10.1001/archderm. 1988.01670060015008.
[35] Feiner, J. R.; Severinghaus, J. W.; Bickler, P. E. Dark Skin Decreases the Accuracy of Pulse Oximeters at Low Oxygen Saturation: The Effects of Oximeter Probe Type and Gender. Anesth. Analg. 2007, 105, S18-S13, doi: 10.1213/01.ane.0000285988.35174.d9.
[36] Bickler, P. E. Effects of Skin Pigmentation on Pulse Oximeter Accuracy at Low Saturation. J. Am. Soc. Anesthesiol. 2005, 102, 715-719.
[37] Parkes, M. J. Breath-Holding and Its Breakpoint. Exp. Physiol. 2006, 91, 1-15, doi: 10.1113/expphysiol.2005.031625.
[38] Glass, K. L.; Dillard, T. A.; Phillips, Y. Y.; Torrington, K. G.; Thompson, J. C. Pulse Oximetry Correction for Smoking Exposure. Mil. Med. 1996, 161, 273-276, doi: 10.1093/milmed/161.5.273.
[39] Chan, E. D.; Chan, M. M.; Chan, M. M. Pulse Oximetry: Understanding Its Basic Principles Facilitates Appreciation of Its Limitations. Respir. Med. 2013, 107, 789-799, doi: 10.1016/j.rmed.2013.02.004.
[40] Chan, M.; Ganti, V. G.; Heller, J. A.; Abdallah, C. A.; Etemadi, M.; Inan, O. T. Enabling Continuous Wearable Reflectance Pulse Oximetry at the Sternum. Biosensors 2021, 11, 521, doi: 10.3390/bios11120521.
[41] de Haan, G.; van Leest, A. Improved Motion Robustness of Remote-PPG by Using the Blood Volume Pulse Signature. Physiol. Meas. 2014, 35, 1913-1926, doi: 10.1088/0967-3334/35/9/1913.
[42] van Gastel, M.; Stuijk, S.; de Haan, G. Motion Robust Remote-PPG in Infrared. IEEE Transactions on Biomedical Engineering 2015, 62, 1425-1433, doi: 10.1109/TBME.2015.2390261.
[43] PCT Patent Publication No. WO2022/115228.

1. A chest-wearable device configured to be attached to or worn in proximity to a person's chest, the wearable device comprising:
a device body having an underside surface;
at least one electrocardiogram (ECG) electrode interface, including a first ECG electrode interface and a second ECG electrode interface, wherein the first ECG electrode interface is located at a first location on the underside surface of the device body, and the second ECG electrode interface is located at a second location on the underside of the device body, each of the first ECG electrode interface and the second ECG electrode interface being configured to operatively couple to a respective patch electrode;
at least one optical sensor configured to obtain Photoplethysmogram waveform acquisitions; and
a plurality of emitters, including a first emitter, a second emitter, and a third emitter, wherein:
the plurality of emitters is configured to emit a light at the person to be received by the at least one optical sensor,
each of the first emitter and the second emitter is configured to operate at a different wavelength configuration for the Photoplethysmogram waveform acquisitions,
the third emitter is employed for artifact identification,
the first emitter, the second emitter, and the third emitter are each positioned at respective location on the underside surface between the first ECG electrode interface and second ECG electrode interface, and
the first ECG electrode interface and second ECG electrode interface, when attached to the respective patch electrodes that are attached to the person, positions the first emitter, the second emitter, and the third emitter proximal to the person for the Photoplethysmogram waveform acquisition.

2. The wearable device of claim 1, wherein at least one of the first emitter, the second emitter, the third emitter, and the at least one optical sensor extends from the underside to position proximally to the person for the Photoplethysmogram waveform acquisition.

3. The wearable device of claim 1 further comprising:
a controller configured, via computer readable instructions or electronic circuitries, to (i) generate a signal quality template of output signals acquired from the third emitter, (ii) generate a signal quality index for a first output signal associated with the first emitter and/or a second output signal associated with the second emitter, wherein the signal quality index for the each of the first and second output signals are employed to reject a portion of the output signals from a peripheral oxygen saturation estimation.

4. The wearable device of claim 1 further comprising:
a controller configured, via computer readable instructions or electronic circuitries, to (i) generate a signal quality template of output signals acquired from the third emitter, (ii) generate a signal quality index for a first output signal associated with the first emitter and/or a second output signal associated with the second emitter, wherein the signal quality index for the each of the first and second output signals are employed to reject a portion of the output signals from a clinical analysis employing the first output signal and the second output signal.

5. The wearable device of claim 1 further comprising:
a controller configured, via computer readable instructions or electronic circuitries, to (i) determine a weight vector of output signals associated with the first, second, and third emitters and (ii) compute a reference signal as a motion-robust AC component for (1) peripheral oxygen saturation estimation or (2) analysis employing the first output signal and the second output signal.

6. The wearable device of claim 5, wherein the weight vector is determined via a matrix operation.

7. The wearable device of claim 1, wherein the first emitter has a center wavelength around a red spectrum.

8. The wearable device of claim 1, wherein the second emitter has a center wavelength around an infrared spectrum.

9. The wearable device of claim 1, wherein the third emitter has a center wavelength around a green spectrum.

10. The wearable device of claim 1, wherein the Photoplethysmogram waveform acquisitions are employed to determine a peripheral oxygen saturation estimation.

11. The wearable device of claim 10 further comprising:
an output display or wireless interface to display, at the output display or an external display, the peripheral oxygen saturation estimation.

12. The wearable device of claim 11, wherein the output display or the wireless interface is configured to display, at the output display or the external display, the electrocardiogram waveform acquisitions from at least one of the first ECG electrode interface and the second ECG electrode interface.

13. The wearable device of claim 1, further comprising an accelerometer configured to measure a seismocardiogram (SCG) signal of the person.

14. The wearable device of claim 10, further comprising:
a controller configured to determine the peripheral oxygen saturation estimation as a ratio of identified alternating current (AC) components and identified direct current (DC) components of the first and second emitter.

15. The wearable device of claim 14, wherein the at least one optical sensor comprises a first optical sensor, wherein the first optical sensor is positioned at the underside surface and proximal to the plurality of emitters.

16. A method to determine pulmonary wedge pressure (PCWP), and pulmonary artery pressure (PAP), the method comprising:
providing a wearable device configured to be attached to or worn in proximity to a person's chest, the wearable device comprising:
a device body having an underside surface;
at least one electrocardiogram (ECG) electrode interface, including a first ECG electrode interface and a second ECG electrode interface, wherein the first ECG electrode interface is located at a first location on the underside surface of the device body, and the second ECG electrode interface is located at a second location on the underside of the device body, each of the first ECG electrode interface and the second ECG electrode interface being configured to operatively couple to a respective patch electrode;
at least one optical sensor configured to obtain Photoplethysmogram waveform acquisitions; and
a plurality of emitters, including a first emitter, a second emitter, and a third emitter, wherein:
the plurality of emitters is configured to emit a light at the person to be received by the at least one optical sensor,
each of the first emitter and the second emitter is configured to operate at a different wavelength configuration for the Photoplethysmogram waveform acquisitions,
the third emitter is employed for artifact identification,
the first emitter, the second emitter, and the third emitter are each positioned at respective location on the underside surface between the first ECG electrode interface and second ECG electrode interface, and
the first ECG electrode interface and second ECG electrode interface, when attached to the respective patch electrodes that are attached to the person, positions the first emitter, the second emitter, and the third emitter proximal to the person for the Photoplethysmogram waveform acquisition;
measuring, a value for the PCWP using the wearable device placed on the person; and
measuring a value for PAP of the person using the wearable device.

17. The method of claim 16, wherein the ECG electrode interface provides a measurement of an ECG signal, the method further comprising:
measuring, by an accelerometer, a SCG signal of the person;
measuring, through a combination of emitters and optical sensors, a photoplethysmogram (PPG) signal of the person; and
estimating the value for the PCWP and/or the value for the PAP using the ECG, SCG, and/or PPG signal.

18. The method of claim 16, further comprising:
tracking changes in the value for the PCWP and/or the value for the PAP using features from SCG, ECG, and PPG signals.

19. The method of claim 17 further comprising:
measuring environmental parameters, including at least one of altitude, humidity, and temperature; and
triggering an estimate for or contextualizing the estimate of the value for the PCWP and/or the value for the PAP using the ECG signal, the SCG signal, and at least one of the altitude, humidity, and temperature.
